SEQUENCE LISTING (6 sequences)

SEQ ID NO: 1 - protein, 249 aa, Homo sapiens
MEMGRRIHLELRNRTPSDVKELVLDNSRSNEGKLEGLTDEFEELEFLSTINVGLTSIANLPKLNKLKKLELSDNRVSGGLEVLAEKCPNLTHLNLSGNKIKDLSTIEPLKKLENLKSLDLFNCEVTNLNDYRENVFKLLPQLTYLDGYDRDDKEAPDSDAEGYVEGLDDEEEDEDEEEYDEDAQVVEDEEDEDEEEEGEEEDVSGEEEEDEEGYNDGEVDDEEDEEELGEEERGQKRKREPEDEGEDDD

SEQ ID NO: 2 - DNA, 1136 bp, Homo sapiens
cgggtgctgggggctcgagaaccgagcggagctggttgagccttcaaagtcctaaaacgcgcggccgtgggttcggggtttattgattgaattccgccggcgcgggagcctctgcagagagagagcgcgagagatggagatgggcagacggattcatttagagctgcggaacaggacgccctctgatgtgaaagaacttgtcctggacaacagtcggtcgaatgaaggcaaactcgaaggcctcacagatgaatttgaagaactggaattcttaagtacaatcaacgtaggcctcacctcaatcgcaaacttaccaaagttaaacaaacttaagaagcttgaactaagcgataacagagtctcagggggcctggaagtattggcagaaaagtgtccgaacctcacgcatctaaatttaagtggcaacaaaattaaagacctcagcacaatagagccactgaaaaagttagaaaacctcaagagcttagaccttttcaattgcgaggtaaccaacctgaacgactaccgagaaaatgtgttcaagctcctcccgcaactcacatatctcgacggctatgaccgggacgacaaggaggcccctgactcggatgctgagggctacgtggagggcctggatgatgaggaggaggatgaggatgaggaggagtatgatgaagatgctcaggtagtggaagacgaggaggacgaggatgaggaggaggaaggtgaagaggaggacgtgagtggagaggaggaggaggatgaagaaggttataacgatggagaggtagatgacgaggaagatgaagaagagcttggtgaagaagaaaggggtcagaagcgaaaacgagaacctgaagatgagggagaagatgatgactaagtggaataacctattttgaaaaattcctattgtgatttgactgtttttacccatatcccctctcccccccccctccaatcctgccccctgaaacttatttttttctgattgtaacgttgctgtgggaacgagaggggaagagtgtactgggggttgcggggggagggatggcgggtgggggtggaataaaatactatttttactgccactctttaaaaaaaaaaaaaaaaaaaaaaaaaaaaaaaaaaaaaaa

SEQ ID NO: 3 - protein, 54 aa, Homo sapiens
KKLELSDNRVSGGLEVLAEKCPNLTHLNLSGNKIKDLSTIEPLKKLENLKSLDL

SEQ ID NO: 4 - DNA, 160 bp, Homo sapiens
tcctggacaacagtcggtcgaatgaaggcaaactcgaaggcctcacagatgaatttgaagaactggaattcttaagtacaatcaacgtaggcctcacctcaatcgcaaacttaccaaagttaaacaaacttaagaagcttgaactaagcgataacagagt

SEQ ID NO: 5 - protein, 195 aa, Homo sapiens
MEMGRRIHLELRNRTPSDVKELVLDNSRSNEGKLEGLTDEFEELEFLSTINVGLTSIANLPKLNKLFNCEVTNLNDYRENVFKLLPQLTYLDGYDRDDKEAPDSDAEGYVEGLDDEEEDEDEEEYDEDAQVVEDEEDEDEEEEGEEEDVSGEEEEDEEGYNDGEVDDEEDEEELGEEERGQKRKREPEDEGEDDD

SEQ ID NO: 6 - DNA, 976 bp, Homo sapiens
cgggtgctgggggctcgagaaccgagcggagctggttgagccttcaaagtcctaaaacgcgcggccgtgggttcggggtttattgattgaattccgccggcgcgggagcctctgcagagagagagcgcgagagatggagatgggcagacggattcatttagagctgcggaacaggacgccctctgatgtgaaagaacttgctcagggggcctggaagtattggcagaaaagtgtccgaacctcacgcatctaaatttaagtggcaacaaaattaaagacctcagcacaatagagccactgaaaaagttagaaaacctcaagagcttagaccttttcaattgcgaggtaaccaacctgaacgactaccgagaaaatgtgttcaagctcctcccgcaactcacatatctcgacggctatgaccgggacgacaaggaggcccctgactcggatgctgagggctacgtggagggcctggatgatgaggaggaggatgaggatgaggaggagtatgatgaagatgctcaggtagtggaagacgaggaggacgaggatgaggaggaggaaggtgaagaggaggacgtgagtggagaggaggaggaggatgaagaaggttataacgatggagaggtagatgacgaggaagatgaagaagagcttggtgaagaagaaaggggtcagaagcgaaaacgagaacctgaagatgagggagaagatgatgactaagtggaataacctattttgaaaaattcctattgtgatttgactgtttttacccatatcccctctcccccccccctccaatcctgccccctgaaacttatttttttctgattgtaacgttgctgtgggaacgagaggggaagagtgtactgggggttgcggggggagggatggcgggtgggggtggaataaaatactatttttactgccactctttaaaaaaaaaaaaaaaaaaaaaaaaaaaaaaaaaaaaaaa

(19) United States
(12) Patent Application Publication
(10) Pub. No.: US 2007/0141062 A1
(43) Pub. Date: Jun. 21, 2007

(54) Control of proliferation and apoptosis in cancer cells

(76) Inventors: Gary R. Pasternack, Baltimore, MD (US); Onikepe Adegbola, Baltimore, MD (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/413,082

(22) Filed: Apr. 28, 2006

Related U.S. Application Data
(60) Provisional application No. 60/675,565, filed on Apr. 28, 2005.

(51) Int. Cl. A61K 39/395; A61K 39/00

(52) U.S. Cl. 424/155.1; 424/185.1

(57) ABSTRACT

The invention includes a method of inhibiting or reducing cellular proliferation through the use of one or more agents that prevent the association or interaction of pp32 polypeptides with the hyperphosphorylated form of Retinoblastoma protein. The invention also discloses agents that are useful in preventing the association or interaction of pp32 polypeptides with the hyperphosphorylated form of Retinoblastoma protein. The invention also discloses screening assays that utilize pp32 polypeptide fragments to identify candidate agents useful in preventing the association or interaction of pp32 polypeptides with the hyperphosphorylated form of Retinoblastoma protein. The invention further discloses diseases and/or disorders for which the disclosed compositions and methods are useful.

### CROSS-REFERENCE TO RELATED PATENT APPLICATIONS

This patent application claims the benefit of U.S. provisional patent application No. 60/675,565, filed Apr. 28, 2005, the disclosure of which is herein incorporated by reference in its entirety.

### BACKGROUND OF THE INVENTION

1. Field of the Invention

This invention pertains to inhibiting or reducing cellular proliferation through the use of agents that prevent the association of the pp32 and the hyperphosphorylated Retinoblastoma proteins.

2. Description of Related Art

The retinoblastoma protein (Rb) is a nuclear phosphoprotein that regulates proliferation, differentiation, and apoptosis. As a tumor suppressor, Rb inhibits proliferation by repressing E2 μl mediated transcription when hypophosphorylated. Hyperphosphorylation of Rb relieves E2 μl repression and allows cell cycle progression to occur (1). The importance of Rb is underscored by the fact that Rb function is disrupted in virtually all human cancers (2). Paradoxically, and inconsistent with its role as a tumor suppressor, hyperphosphorylated wild-type Rb inhibits apoptosis in both cell culture and animal models (3-10). Since Rb inactivation is pivotal for carcinogenesis, this poses the problem of how cancer cells escape apoptosis when Rb function is disrupted.

Inherited cancers and cells in which Rb is inactivated by mutation have increased rates of both proliferation and apoptosis (11). Most sporadic cancers preferentially inactivate Rb by hyperphosphorylation, which may occur through mutation of cyclin D, cdk4 or p16. Such cancers are generally slow growing and resistant to apoptosis induced by chemotherapy or radiation (12). It is possible that in these cancers the tumor suppressor function of Rb is inhibited while the antiapoptotic function remains intact (13). Inactivation by hyperphosphorylation might promote proliferation by increasing free E2F1, as well as inhibit apoptosis by retaining the anti-apoptotic function of Rb. This is consistent with evidence suggesting that it is the hyperphosphorylated form of Rb rather than Rb per se that protects cells from apoptosis (14). The induction of apoptosis in various cell lines is accompanied by a shift in Rb from the hyper- to the hypo-phosphorylated form (15, 16). Rb dephosphorylation, which has been shown to be required for apoptosis, occurs in the early stage of apoptosis (17, 18). Inhibition of Rb dephosphorylation prevents apoptosis, while induction of dephosphorylation leads to apoptosis (19). In DBA/2 mice increased levels of hyperphosphorylated Rb appear to mediate apoptotic resistance (20). An increased level of hyperphosphorylated Rb is associated with a worse clinical outcome and greater chemoresistance as compared to Rb loss or normal levels of unphosphorylated Rb in patients with anaplastic large cell lymphoma (ALCL) (21). These observations all point to a pivotal role for hyperphosphorylated Rb in inhibiting apoptosis.

Although the exact mechanism by which Rb inhibits apoptosis is unclear, it does not always require inhibition of E2F1-mediated transcription by Rb. The increase in apoptosis seen in Rb-null embryos is only partially reversed in Rb and E2F1 double knockouts (22). A caspase-resistant Rb mutant (Rb-MI) inhibited apoptosis in response to TNF alpha induced apoptosis by interfering with caspase 3 activation (23). These results led to the postulate that Rb binds to and sequesters a nuclear factor that stimulates caspase 3 activation (24). Although Rb binds to over 100 protein partners, the majority bind to the hypophosphorylated form (25), yet it is the hyperphosphorylated form that predominates in most sporadic cancers.

pp32 is a member of the ANP32 family of acidic, leucine-rich nuclear phosphoproteins found in cells capable of self-renewal and in certain long-lived neuronal populations (26). pp32 has been referred to variously throughout the scientific literature as PHAPI, LANP, I1PP2a, and mapmodulin, but all of these names refer to the product of the ANP32A gene. pp32 has been implicated in a number of cellular processes, including proliferation (27), differentiation (28), caspase dependent and caspase independent apoptosis (29, 30), suppression of transformation in vivo (31, 32), inhibition of protein phosphatase 2A (33), regulation of mRNA trafficking and stability in association with HuR (34), and inhibition of acetyltransferases as part of the INHAT complex (35).

At a biologic level, pp32 inhibits transformation of rat embryo fibroblasts (36), possibly through its pro-apoptotic activity. It accelerates caspase activation by stimulating the apoptosome, but the in vivo significance of this is unclear. In contradistinction to its pro-apoptotic and transformation inhibition functions, pp32 is highly expressed in cancer (37). In fact, pp32 is more highly expressed in highly malignant prostatic adenocarcinomas of Gleason score ≧5 than in clinically indolent tumors of Gleason score <5 (38). These data suggest that high levels of pp32 might foster increased malignancy. We report here that hyperphosphorylated Rb and pp32 associate in a specific complex. The pp32-Rb interaction inhibits the apoptotic activity of pp32 and promotes increased proliferation.

There remains a need in the art for compositions and methods useful for controlling proliferative growths in patients in need of such treatment, such as patients suffering from cancerous growths. Compositions or methods that reduce or inhibit the proliferation of unwanted cells, such as cancerous cells, are beneficial to the patient in need thereof.

### BRIEF SUMMARY OF THE INVENTION

The invention includes a method of reducing the proliferation of cells, such as cancerous cells, in a subject in need thereof. The invention includes a method of reducing the proliferation of cells in a subject by administering at least one agent that inhibits or prevents the association of the pp32 and hyperphosphorylated Retinoblastoma (pRb) proteins.

The invention also includes agents that are useful for inhibiting or preventing the association of the pp32 and pRb proteins. Such agents include, but are not limited to, peptides that have binding specificity for pRb, such as peptides comprising, or alternatively consisting of, a peptide sequence corresponding to amino acids 67 to 120 of human pp32 protein, as well as fragments and variants thereof that retain the binding specificity for the pRb protein and that inhibit or prevent the association of the pp32 and pRb proteins.

The invention also includes screening assays designed to identify those candidate agents capable of inhibiting the association of pp32 with pRb. In one embodiment of the invention, the assay comprises mixing a candidate agent with pp32 and pRb proteins, and measuring the binding of pp32 to pRb in the presence of the candidate agent.

### BRIEF DESCRIPTION OF THE SEVERAL VIEWS OF THE DRAWING(S)

The invention is further described in the following Figures, in which:

FIG. 1 illustrates that pp32 interacts with Rb via an LRR motif.

(A) Rb co-immunoprecipitates with V5-tagged pp32. HEK 293 cells were transfected with pp32V5. Equal amounts of cell extracts were precipitated with anti-V5 or a control (NSE) antibody and the presence of Rb in the immunoprecipitates was visualized by western blot analysis using anti-Rb (G3-245) antibody.

(B) V5-tagged pp32 co-immunoprecipitates with Rb. HEK 293 cells were transfected with pp32V5 and lacZV5 as indicated. Equal amounts of cell extracts were precipitated with anti-Rb (G99-2005) antibody and the presence of V5 in immunoprecipitates was visualized by western blot analysis using anti-V5 antibody.

(C) Endogenous pp32 co-immunoprecipitates with endogenous Rb. Equal amounts of HeLa cell extracts were precipitated with anti-Rb (G99-2005) or a control (PCNA) antibody and the presence of pp32 in immunoprecipitates was visualized by western blot analysis using anti-pp32 antibody. Identical results were obtained with HEK 293 and K562 cells

(D) Schematic diagram of pp32 mutants. Nucleic acid number of domain boundaries is indicated. All contain a COOH-terminal V5 epitope tag.

(E) Rb interacts with the LRR of pp32. HEK 293 cells were transfected with the indicated V5 epitope tagged mutants. In both panels, the unlabeled lane on the left represents molecular weight markers of 20, 30, and 40 kDa. Upper panel, equal amounts of cell extracts were precipitated with anti-Rb (G99-2005) antibody and the presence of V5 in immunoprecipitates was visualized by western blot analysis using anti-V5 antibody. The arrow indicates the position of immunoglobulin light chain. Lower panel, cell extracts were subjected to anti-V5 western blot analysis to confirm expression of the indicated V5 epitope tagged mutants.

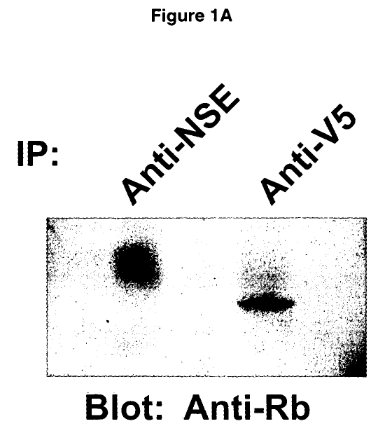

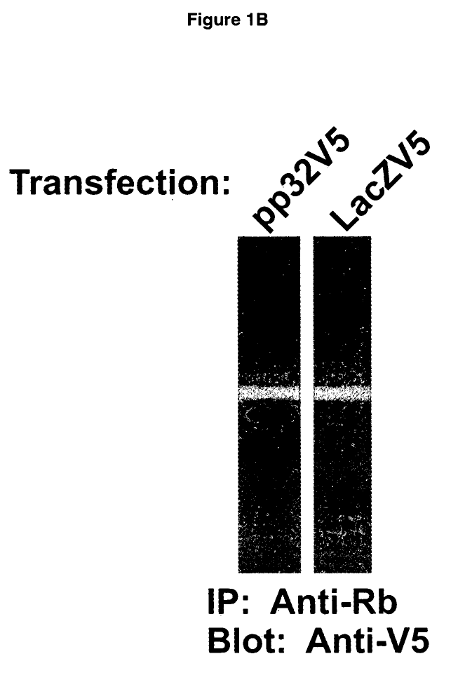

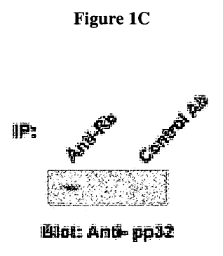

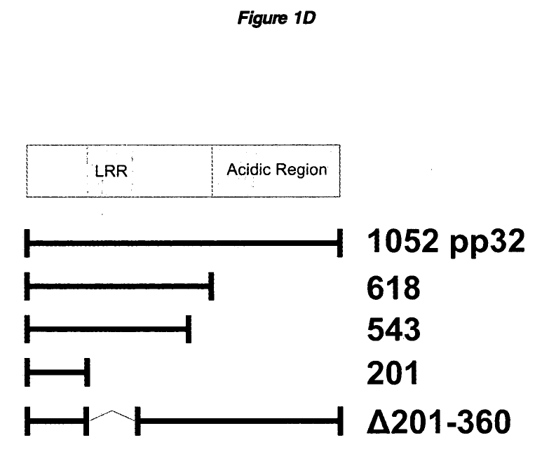

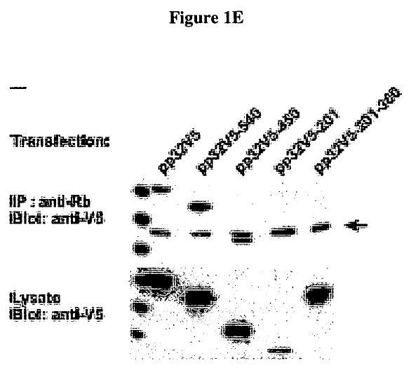

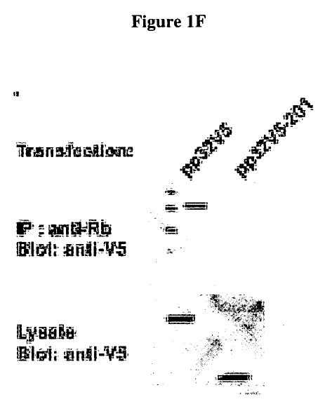

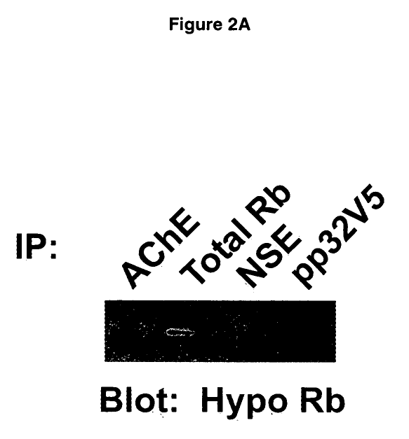

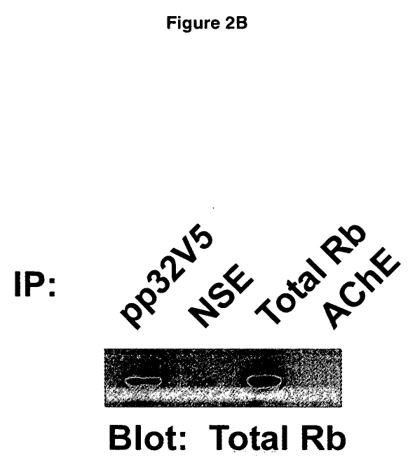

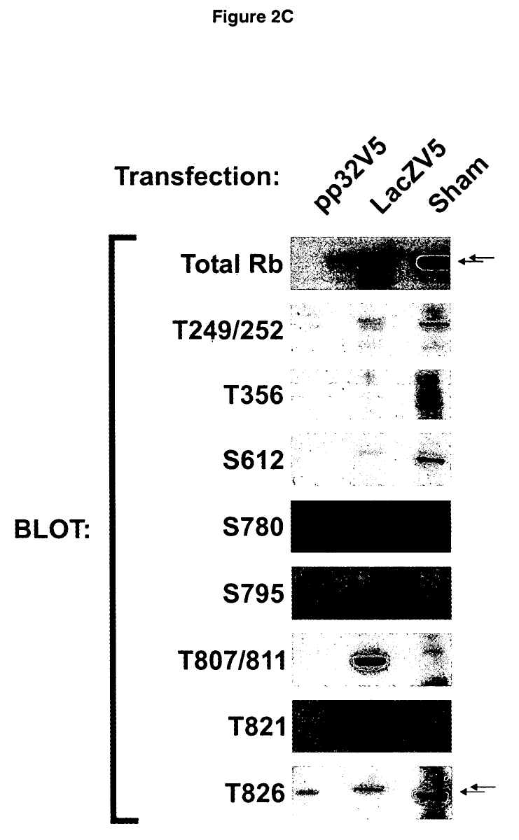

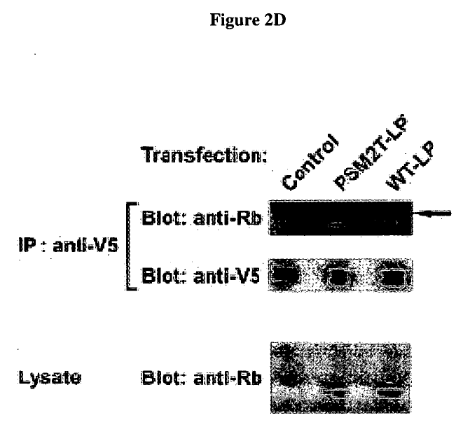

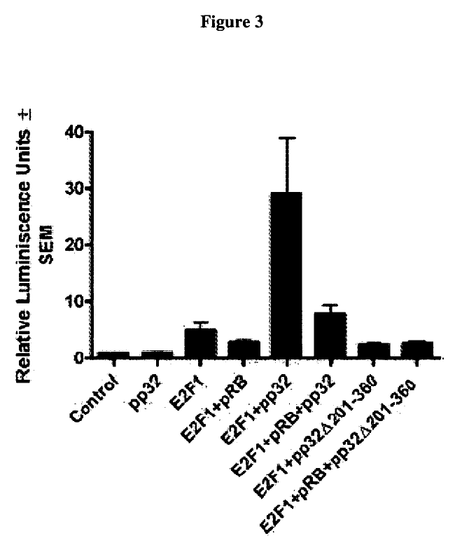

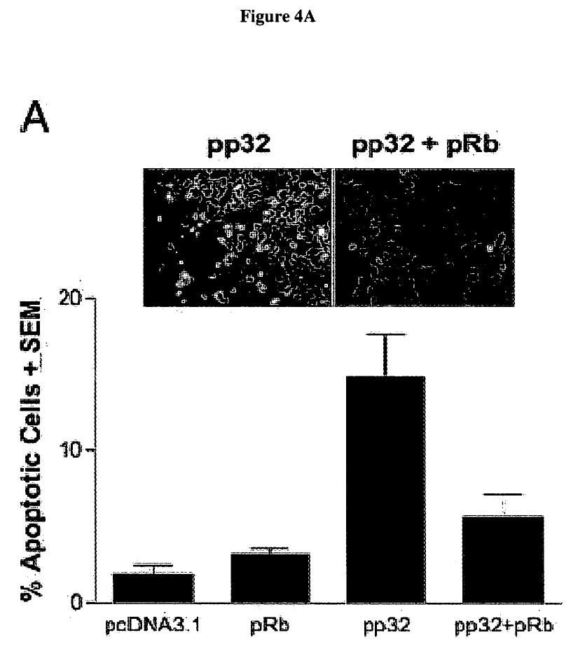

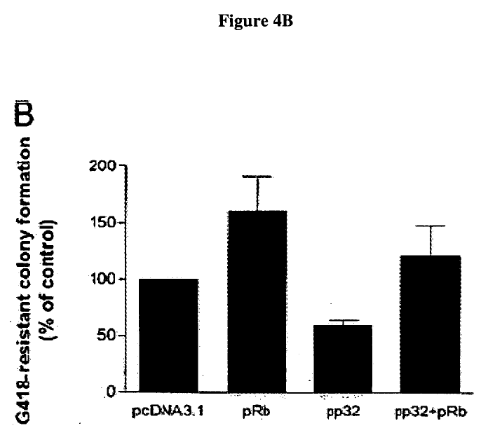

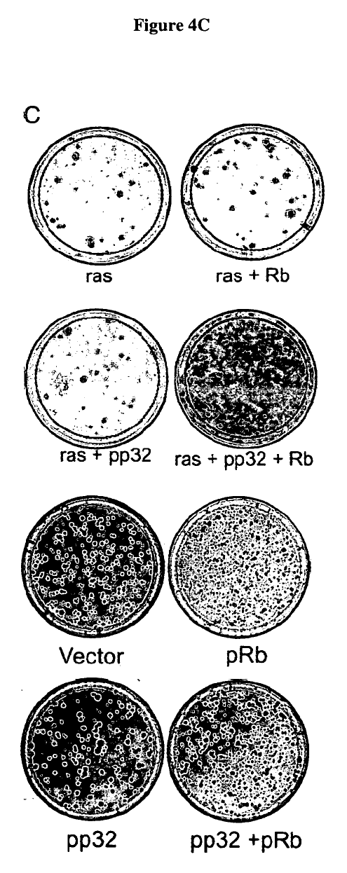

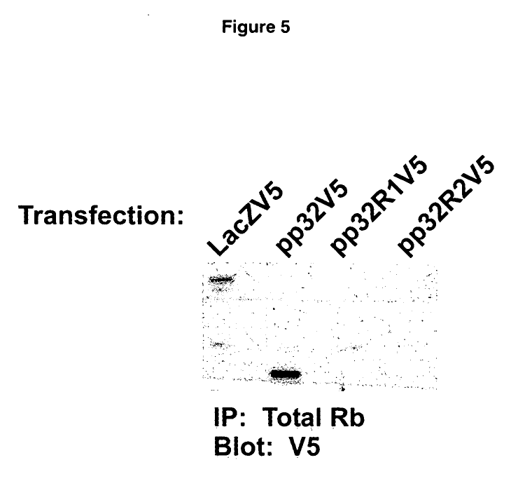

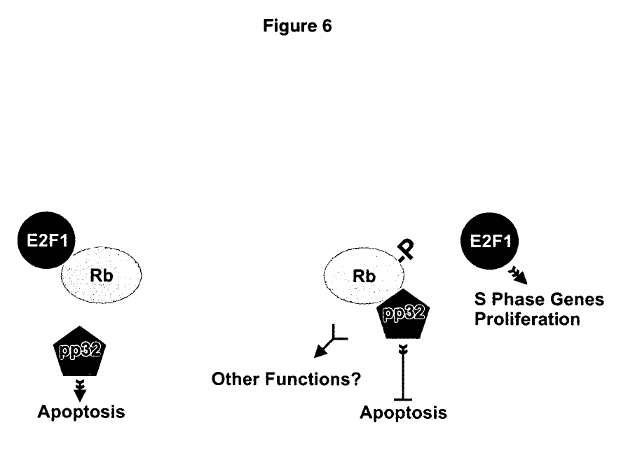

(F) Replicate of the experiment shown in FIG. 1E restricted to pp32V5 and pp32V5-201 at a higher level of expression.

FIG. 2 illustrates that pp32 binds preferentially to Rb phosphorylated on T⁸²⁶.

(A) HEK 293 cells were transfected with pp32V5 or sham transfected. Equal amounts of transfected cell extracts were precipitated with anti-V5 or a control (NSE) antibody as indicated. Equal amounts of sham transfected cell extracts were precipitated with total Rb (C-15) or control (AChE) antibody as indicated. The presence of hypophosphorylated Rb in the immunoprecipitates was analysed by immunoblotting using an antibody specific for hypophosphorylated Rb (G99-549).

(B) The blot in (A) was stripped and re-probed with total Rb (G3-245) antibody.

(C) HEK 293 cells were transfected with pp32V5, LacZV5 or sham transfected as indicated. Equal amounts of pp32V5 and LacZV5 transfected cell extracts were precipitated with anti-V5. As a positive control, sham transfected cell extracts were precipitated with antibody to total Rb (G3-245); this control, which precipitates considerably more Rb, is designated “Sham” on the figure. The presence of specific phosphorylated forms of Rb in the immunoprecipitates was analysed by immunoblotting using the indicated anti-phospho-Rb antibodies.

(D) HEK 293 cells were cotransfected with the pp32V5 and control, WT-LP or PSM2T-LP as indicated. Equal amounts of cell extracts were precipitated with anti-V5 antibody. Upper panel, the presence of WT-LP or PSM2T-LP in immunoprecipitates was probed by western blot analysis using anti-Rb antibody (851). The right-hand arrow indicates the position of the Rb large pocket fragment. The lower band present in all three lanes is immunoglobulin heavy chain, which serves as a loading control. Middle panel, anti-V5 immunoprecipitates were subjected to western blot analysis with anti-V5 antibody to confirm pp32V5 immunoprecipitation. Lower panel, cell extracts were subjected to western blotting with anti-Rb antibody (851) to confirm expression of WT-LP and PSM2T-LP.

FIG. 3 illustrates that pp32 increases E2F1-mediated transcriptional activity.

(A) NIH 3T3 cells were transiently transfected with E2F-luciferase reporter vector (pE2F-TA-Luc) and, where indicated, E2F1, pRb, pp32V5 or pp32Δ201-360V5 expression vectors. Data are presented as the mean±SEM from three independent experiments performed in duplicate.

FIG. 4 illustrates that the association between Rb and pp32 correlates with inhibition of pp32 apoptotic activity.

pp32 induced apoptosis is abrogated by Rb in mammalian cells. HeLa cells were transfected with expression plasmids encoding the indicated proteins. Nuclei were stained with Hoechst stain and examined by immunofluorescence microscopy for characteristics of apoptosis (membrane blebbing, chromatin condensation and pyknosis). Cell death was quantified in HeLa cells transfected with the indicated expression constructs. The data (mean±SEM) are the percentage of nuclei counted with apoptotic morphology (n equals at least three experiments).

(B) Duplicate plates of HeLa cells were transfected with control, pp32, or pRb expression plasmids as indicated and subjected to colony formation assay. Plates were stained with methylene blue and total number of G418 resistant colonies were counted after 14 days of selection. A representative experiment is shown. The bar graph (mean±SEM) shows the percentage change in colony formation efficiency with the colony counts normalized against the vector only control (n equals at least three experiments in duplicate).

(C) Duplicate plates of NIH 3T3 cells were transfected with ras, pp32, or pRb expression plasmids as indicated and subjected to colony formation assay. Plates were stained with methylene blue and photographed after 14 days of G418 selection.

FIG. 5 illustrates that Rb associates with pp32, but not with other members of the ANP32 family. HEK 293 cells were transfected with pp32V5, pp32r1V5, pp32r2V5 or LacZV5 expression vectors as indicated. Equal amounts of cell extracts were precipitated with anti-Rb (G99-2005) antibody and analysed by western blotting with anti-V5 antibody.

FIG. 6 illustrates a non-limiting, proposed model of pp32-Rb interaction. In normal cells, pp32 overexpression results in apoptosis. In cancer cells, high levels of hyperphosphorylated Rb sequester pp32, leading to inhibition of apoptosis, increased free E2F1 and increased proliferation.

### DETAILED DESCRIPTION OF PREFERRED EMBODIMENTS

Definitions

As used herein, “hyperphosphorylated Rb” (pRb) is intended to mean pRb that exhibits a modified gel shift profile compared to the hypophosphorylated form of the Retinoblastoma protein. One of skill in the art understands what is intended through the use of “hyperphosphorylated Rb”, as evidenced by the review article of Sibylle Mittnacht, Current Opinion in Genetics & Development, 8:21-27 (1998), which is incorporated herein by reference in its entirety, as well as the references cited by this review article, which are also incorporated herein by reference in their entireties.

As used herein, “association between pp32 and pRb” is intended to mean the direct binding, or the indirect binding through the formation of a protein complex, of the pp32 and pRb proteins.

As used herein, “prevents the association” is intended to encompass instances where formation of an association between pp32 and pRb is prevented, or made weaker or an existing association of pp32 and pRb is disrupted by the presence of one or more agents as set forth in the invention.

As used herein, “binding peptide” is intended a peptide that preferentially binds directly to, or binds indirectly through the formation of a protein complex, to a particular, identifiable binding partner, usually a protein such as the pRb protein. Similarly, the recitation of “binding specificity” for pRb is intended to signify that polypeptides such as peptides or antibody fragments may bind directly to, or indirectly to through the formation of a protein complex, the pRb protein.

As used herein, “promoting apoptosis” in intended to mean that which affects a cell population so that the fraction of cells in the population undergoing apoptosis increases detectably. Methods for detecting cells undergoing apoptosis, and for quantifying them, are well known in the art. Such methods include examination of nuclear morphology as described, e.g., in Example 8 below, or TUNEL assay, as described by Negoescu, et al., in J. Histochem. Cytochem., 44:959-968 (1996), incorporated herein by reference.

In discussing the structure of particular double-stranded polynucleotide molecules, sequences may be described herein according to the normal convention of giving only the sequence in the 5′ to 3′ direction along the nontranscribed strand of DNA (i.e., the strand having a sequence homologous to the mRNA).

A polynucleotide sequence “corresponds” to a polypeptide sequence if translation of the polynucleotide sequence in accordance with the genetic code yields the polypeptide sequence (i.e., the polynucleotide sequence “encodes” the polypeptide sequence), one polynucleotide sequence “corresponds” to another polynucleotide sequence if the two sequences encode the same polypeptide sequence.

Two polynucleotide sequences are “substantially similar” when at least about 90% (preferably at least about 94%, and most preferably at least about 96%) of the nucleotides match over the defined length of the DNA sequences. Sequences that are substantially similar can be identified by the assay procedures described below or by isolating and sequencing the polynucleotide molecules. See e.g., Maniatis et al., infra, DNA Cloning, vols. I and II infra: Nucleic Acid Hybridization, infra.

A “heterologous” region or domain of a DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A “coding sequence” or “open reading frame” is an in-frame sequence of codons that (in view of the genetic code) correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptides in vivo. A polyadenylation signal and transcription termination sequence will usually be located 3′ to the coding sequence. A “promoter sequence” is a DNA regulatory region capable of binding RNA polymerase in a cell and initiating transcription of a downstream (3′ direction) coding sequence. Promoter sequences typically contain additional sites for binding of regulatory molecules (e.g., transcription factors) which affect the transcription of the coding sequence. A coding sequence is “under the control” of the promoter sequence or “operatively linked” to the promoter when RNA polymerase binds the promoter sequence in a cell and transcribes the coding sequence into mRNA, which is then in turn translated into the protein encoded by the coding sequence.

Vectors are used to introduce a foreign substance, such as DNA, RNA or protein, into an organism. Typical vectors include recombinant viruses (for polynucleotides) and liposomes (for polypeptides). A “DNA vector” is a replicon, such as plasmid, phage or cosmid, to which another polynucleotide segment may be attached so as to bring about the replication of the attached segment. An “expression vector” is a DNA vector which contains regulatory sequences which will direct polypeptide synthesis by an appropriate host cell. This usually means a promoter to bind RNA polymerase and initiate transcription of mRNA, as well as ribosome binding sites and initiation signals to direct translation of the mRNA into a polypeptide(s). Incorporation of a polynucleotide sequence into an expression vector at the proper site and in correct reading frame, followed by transformation of an appropriate host cell by the vector, enables the production of a polypepide encoded by said polynucleotide sequence.

An expression vector may alternatively contain an antisense sequence, where a small polynucleotide fragment, corresponding to all or part of an mRNA sequence, is inserted in opposite orientation into the vector after a promoter. As a result, the inserted polynucleotide will be transcribed to produce an RNA which is complementary to and capable of binding or hybridizing with the mRNA. Upon binding to the mRNA, translation of the mRNA is prevented, and consequently the protein coded for by the mRNA is not produced. Production and use of antisense expression vectors is described in more detail in U.S. Pat. No. 5,107,065 (describing and exemplifying antisense regulation of genes in plants) and U.S. Pat. No. 5,190,931 (describing antisense regulation of genes in both prokaryotes and eukaryotes and exemplifying prokaryotes), both of which are incorporated herein by reference.

“Amplification” of polynucleotide sequences is the in vitro production of multiple copies of a particular nucleic acid sequence. The amplified sequence is usually in the form of DNA. A variety of techniques for carrying out such amplification are described in a review article by Van Brunt (1990, Bio/Technol., 8(4):291-294). Polymerase chain reaction or PCR is a prototype of nucleic acid amplification, and use of PCR herein should be considered exemplary of other suitable amplification techniques.

For the purposes of defining the present invention, two polypeptides are homologous if 80% of the amino acids in their respective amino acid sequences are the same; for proteins of differing length, the sequences will be at least 80% identical over the sequence which is in common (i.e., the length of the shorter protein).

Two amino acid sequences are “substantially similar” when at least about 87% of the amino acids match over the defined length of the amino acid sequences, preferably a match of at least about 89%, more preferably a match of at least about 95%. Typically, two amino acid sequences which are similar will differ by only conservative substitutions.

“Conservative amino acid substitutions” are the substitution of one amino acid residue in a sequence by another residue of similar properties, such that the secondary and tertiary structure of the resultant peptides are substantially the same. Conservative amino acid substitutions occur when an amino acid has substantially the same charge or hydrophobicity as the amino acid for which it is substituted and the substitution has no significant effect on the local conformation of the protein. Amino acid pairs which may be conservatively substituted for one another are well-known to those of ordinary skill in the art.

As contemplated herein, peptides of this invention include oligopeptides, polypeptides and proteins. The polypeptides of this invention encompass pp32 analogs, fragments thereof, other variants of pp32 and their analogs, as well as including forms of heterogeneous molecular weight that may result from inconsistent processing in vivo. Analogs of peptides contemplated herein include amino acid polymers that comprise D amino acids and amino acids linked by one or more non-peptide bonds, so long as the analog retains the ability to inhibit or prevent the association of pp32 and pRb. An example of the pp32 sequence is shown in SEQ ID NO:1. pp32 polypeptides include, but are not limited to:

1) “Mutants or Variants of pp32,” which are polypeptides which are substantially similar to pp32 and retain the ability to inhibit the association of pp32 with pRb;

2) “Truncated pp32 peptides.” which include fragments of pp32 that preferably retain the ability to inhibit or prevent the association of pp32 with pRb;

3) “pp32 fusion proteins,” which include heterologous polypeptides which are made up of one of the above polypeptides (pp32 or truncated pp32 polypeptides, as well as mutants and variants thereof) fused to any heterologous amino acid sequence.

“Variants of pp32” are homologous proteins which differ from pp32 by at least 2 amino acids, but that retains the ability to inhibit the association of pp32 with pRb. Such variants include, but are not limited to deletions, additions and substitutions in the amino acid sequence of the polypeptide peptide. For example, one class of substitutions are conserved amino acid substitution. Such substitutions are those that substitute a given amino acid in a polypeptide by another amino acid of like characteristics. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg; and replacements among the aromatic residues Phe and Tyr. Guidance concerning which amino acid changes are likely to be phenotypically silent are found in Bowie et al. (1990) Science 247: 1306-1310 which is incorporated by reference.

A composition comprising a selected component A is “substantially free” of another component B when component A makes up at least about 75% by weight of the combined weight of components A and B. Preferably, selected component A comprises at least about 90% by weight of the combined weight, most preferably at least about 99% by weight of the combined weight. In the case of a composition comprising a selected biologically active protein, which is substantially free of contaminating proteins, it is sometimes preferred that the composition having the activity of the protein of interest contain species with only a single molecular weight (i.e., a “homogeneous” composition).

As used herein, a “biological sample” refers to a sample of tissue or fluid isolated from a individual, including but not limited to, for example, plasma, serum, spinal fluid, lymph fluid, the external sections of the skin, respiratory, intestinal, and genitourinary tracts, tears, saliva, milk, blood cells, tumors, organs, and also samples of in vivo cell culture constituents (including but not limited to conditioned medium resulting from the growth of cells in cell culture medium, putatively virally infected cells, recombinant cells, and cell components).

A cell population as contemplated herein includes a suspension of cells in a fluid medium as well as an aggregate of cells which constitute a solid mass or a tissue. Such a cell population may be a constituent of a biological organism. When the cell population is part of an organism, “administration” to the organism will encompass administration to the cell population. When the cell population is a suspension in a fluid medium, then adding a component to the medium will result in “administration” of that component to the cells.

“Human tissue” is an aggregate of human cells which may constitute a solid mass. This term also encompasses a suspension of human cells, such as blood cells, or a human cell line.

The term “antibody” encompasses whole antibodies made up of four immunoglobulin peptide chains, two heavy chains and two light chains, as well as immunoglobulin fragments. “Antibody fragments” are protein molecules related to antibodies, which are known to retain the epitopic binding specificity of the original antibody, such as Fab, F(ab)′₂, Fv, etc. Two polypeptides are “immunologically cross-reactive” when both polypeptides react with the same polyclonal antiscrum.

Methods of Inhibiting the Association of pp32 and pRb

The invention includes a method of reducing the proliferation of cells, such as cancerous cells, in a subject in need thereof. The invention includes a method of reducing the proliferation of cells in a subject by administering at least one agent that inhibits or prevents the association of the pp32 and hyperphosphorylated Retinoblastoma (pRb) proteins.

In one embodiment of the invention, a patient suffering from a proliferative disorder such as cancer, is administered an agent that prevents the association of the pp32 and pRb proteins. In a non-limiting hypothesis of the invention, it is believed that the administration of one or more of said agents prevents the association of pp32 and pRb, thereby permitting the pro-apoptotic activity attributed to the pp32 protein, which is otherwise sequestered by pRb (said sequestering diminishing the pro-apoptotic activity attributed to the pp32 protein).

The methods of the invention are useful in reducing, treating or preventing proliferative disorders such as cancers. In a preferred embodiment of the invention, the methods of the invention are useful in reducing, treating or preventing breast, colon, lung, stomach, and pancreatic cancers, leukemias, lymphomas, melanomas and other skin cancers, and brain cancers including glioblastomas.

Agents for Inhibiting the Association of pp32 and pRb

The invention includes agents that are useful for inhibiting or preventing the association of the pp32 and pRb proteins. Such agents include, but are not limited to, peptides that have binding specificity for pRb, such as peptides comprising, or alternatively consisting of, an isolated peptide sequence corresponding to amino acids 67 to 120 of human pp32 protein, as well as fragments and variants thereof that retain the binding specificity for the pRb protein and that inhibit or prevent the association of the pp32 and pRb proteins.

The polypeptide sequence of the human pp32 protein is provided herein as SEQ ID NO:1. The corresponding polynucleotide sequence encoding the human pp32 protein is provided herein as SEQ ID NO:2. The polypeptide and polynucleotide sequences of the full length human pp32 have also been previously disclosed in numerous publications, including at least U.S. Pat. Nos. 6,040,173 and 6,930,175, the disclosure of which are herein incorporated by reference in their entireties.

In one embodiment of the invention, an agent that is useful for inhibiting or preventing the association of the pp32 and pRb proteins is an isolated polypeptide comprising, or alternatively consisting of, amino acids 67 to 120 of human pp32 and as presented as SEQ ID NO:3. It is contemplated that this agent further comprises mutants and variants, as well as fragments, of the sequence of amino acids 67 to 120 of pp32, provided that the peptide retains the ability to inhibit or prevent the association of the pp32 and pRb proteins. In addition, this agent may also have the activities of increasing pp32-mediated apoptosis and/or of decreasing E2F1-mediated transcription.

In another embodiment of the invention, an agent that is useful for inhibiting or preventing the association of the pp32 and pRb proteins is an isolated polynucleotide encoding a polypeptide comprising, or alternatively consisting of, amino acids 67 to 120 of human pp32. It is contemplated that this agent further encodes a polypeptide comprising mutants and variants, as well as fragments, of the sequence of amino acids 67 to 120 of pp32, provided that the peptide encoding by said polynucleotide retains the ability to inhibit or prevent the association of the pp32 and pRb proteins. The isolated polynucleotide may be contained in an expression vector system or a gene therapy vector system, for expression of the encoded polypeptide in vitro or in vivo. In addition, this agent may also have the activities of increasing pp32-mediated apoptosis and/or of decreasing E2F1-mediated transcription. In vitro expression systems are discussed in greater detail below. The polynucleotide encoding the polypeptide comprising amino acids 67 to 120 of pp32 is presented as SEQ ID NO:4.

In another embodiment of the invention, an agent that is useful for inhibiting or preventing the association of the pp32 and pRb proteins comprises, or alternatively consists of, an isolated antibody fragment having binding specificity for the pRb protein and that has the activity of preventing the association of the pp32 and pRb proteins. One of skill in the art may readily ascertain whether a given antibody or fragment thereof retains the ability to prevent the association of the pp32 and pRb proteins. Exemplary antibody fragments include, but are not limited to, Fab, F(ab)′₂, and Fv having the biological activity of preventing or inhibiting the association of the pp32 and pRb proteins. In a particularly preferred embodiment of the invention, the antibody binding site includes Thr⁸²⁶ of pRb. In addition, this agent may also have the activities of increasing pp32-mediated apoptosis and/or of decreasing E2F1-mediated transcription.

In another embodiment of the invention, an agent that is useful for inhibiting or preventing the association of the pp32 and pRb proteins comprises, or alternatively consists of, an isolated polynucleotide encoding an antibody or a fragment thereof having binding specificity for the pRb protein and that has the activity of preventing the association of the pp32 and pRb proteins. One of skill in the art may readily ascertain whether a given antibody fragment encoded by said polynucleotides retains the ability to prevent the association of the pp32 and pRb proteins. Exemplary antibody fragments that may be encoded by said polynucleotides include, but are not limited to, Fab, F(ab)′₂, and Fv having the biological activity of preventing or inhibiting the association of the pp32 and pRb proteins. The isolated polynucleotide may be contained in an expression vector system or a gene therapy vector system, for expression of the encoded polypeptide in vitro or in vivo. Expression systems are discussed in greater detail below. In addition, this agent may also have the activities of increasing pp32-mediated apoptosis and/or of decreasing E2F1-mediated transcription.

In another embodiment of the invention, polynucleotides encoding polypeptide agents that are useful for inhibiting or preventing the association of the pp32 and pRb proteins may be engineered to be targeted to the nucleus using methods known in the art, such as for example the inclusion of a nuclear localization signal. Nuclear localization signals (NLS) are amino acid sequences which have evolved in polypeptides, thereby facilitating migration of a polypeptide from the cytoplasm into the nucleus. Specified nuclear polypeptides containing NLS domains have been shown to enable the transport of a polypeptide-RNA complex into the nucleus (Mattaj and DeRobertis, 1985).

Non-limiting examples of NLS domains are provided in U.S. Pat. No. 6,720,310, the disclosure of which is herein incorporated by reference in its entirety.

Nuclear localization signals are also commercially available from Invitrogen (Carlsbad, Calif.) using their pShooter™ mammalian expression vectors which incorporate signal sequences into recombinant proteins to direct them to a specific subcellular location. The vectors are available for targeting proteins to the nucleus or mitochondria as well as to the cytoplasm.

In another embodiment of the invention, agents that are useful for inhibiting or preventing the association of the pp32 and pRb proteins comprises, or alternatively consists of, one or more expression systems comprising the polynucleotide sequence of SEQ ID NO:6, which encodes a deletion mutant of the pp32 protein lacking amino acids 67-120 (a dominant negative). The encoded polypeptide lacking amino acids 67-120 is presented as SEQ ID NO:5. In a non-limiting hypothesis of the invention, the dominant negative peptide does not bind to pRb due to the absence of amino acids 67-120. Expression systems are discussed in greater detail below. In addition, this agent may also have the activities of increasing pp32-mediated apoptosis and/or of decreasing E2F1-mediated transcription.

In a further embodiment of the invention, agents that are useful for inhibiting or preventing the association of the pp32 and pRb proteins comprises, or alternatively consists of, a peptidomimetic. Peptidomimetic refers to a synthetic chemical compound which has substantially the same structural and/or functional characteristics as the polypeptides of the invention. The mimetic can be entirely composed of synthetic, non-natural amino acid analogues, or can be a chimeric molecule including one or more natural peptide amino acids and one or more non-natural amino acid analogs. The mimetic can also incorporate any number of natural amino acid conservative substitutions as long as such substitutions do not destroy the activity of the peptidomimetic. As with polypeptides of the invention which are conservative variants, routine testing can be used to determine whether a peptidomimetic has the requisite activity, e.g., that it prevents the association of pp32 with pRb. In addition, a peptidomimetic may also have the activities of increasing pp32-mediated apoptosis and/or of decreasing E2F1-mediated transcription.

In a preferred embodiment of the invention, peptidomimetics of the invention are based on the polypeptide sequence corresponding to amino acids 67 to 120 of pp32 (SEQ ID NO:3).

Non-limiting examples of non-natural residues useful in the production of peptidomimetics of natural amino acid residues are mimetics of aromatic amino acids including, for example, D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acid. Aromatic rings of a non-natural amino acid that can be used in place a natural aromatic rings include, for example, thiazolyl, thiophenyl, pyrazolyl, benzimidazolyl, naphthyl, furanyl, pyrrolyl, and pyridyl aromatic rings.

Peptidomimetics may be synthesized using a variety of procedures and methodologies known in the art (see, e.g., Organic Syntheses Collective Volumes, Gilman, et al. (Eds) John Wiley & Sons, Inc., NY; al-Obeidi, Mol. Biotechnol. 9:205 223 (1998); Hruby, Curr. Opin. Chem. Biol. 1:114, 119 (1997); Ostergaard, Mol. Divers. 3:17, 27 (1997); and Ostresh, Methods Enzymol. 267:220, 234 (1996)).

Inhibitory Agents and Methods of Making

The practice of the present invention employs, unless otherwise indicated, conventional molecular biology, microbiology, and recombinant DNA techniques within the skill of the art. Such techniques are well known to the skilled worker and are explained fully in the literature. See, e.g., Maniatis, Fritsch & Sambrook. “Molecular Cloning: A Laboratory Manual” (1982); “DNA Cloning: A Practical Approach.” Volumes I and II (D. N. Glover, ed., 1985); “Oligonucleotide Synthesis” (M. J. Gait, ed., 1984), “Nucleic Acid Hybridization” (B. D. Hames & S. J. Higgins, eds., 1985): “Transcription and Translation” (B. D. Hames & S. J. Higgins, eds., 1984): “Animal Cell Culture” (R. I. Freshney, ed., 1986); “Immobilized Cells and Enzymes” (IRL Press. 1986); B. Perbal, “A Practical Guide to Molecular Cloning” (1984), and Sambrook, et al., “Molecular Cloning: a Laboratory Manual” (1989).

Polynucleotides

The present invention further provides isolated nucleic acid molecules that encode a polypeptide of the present invention. Such nucleic acid molecules will consist of, consist essentially of, or comprise a nucleotide sequence that encodes one of the polypeptide of the present invention, or an ortholog or paralog thereof. As used herein, an “isolated” nucleic acid molecule is one that is separated from other nucleic acid present in the natural source of the nucleic acid.

Isolated polynucleotides or oligonucleotides having specific sequences can be synthesized chemically or isolated by one of several approaches. The basic strategies for identifying, amplifying and isolating desired polynucleotide sequences as well as assembling them into larger polynucleotide molecules containing the desired sequence domains in the desired order, are well known to those of ordinary skill in the art. See, e.g., Sambrook, et al., (1989); B. Perbal. (1984). Preferably, polynucleotide segments corresponding to all or a part of the cDNA or genomic sequence of pp32 may be isolated individually using the polymerase chain reaction (M. A. Innis, et al., “PCR Protocols: A Guide To Methods and Applications.” Academic Press. 1990). A complete sequence may be assembled from overlapping oligonucleotides prepared by standard methods and assembled into a complete coding sequence. See, e.g., Edge (1981) Nature 292:756; Nambair, et al. (1984) Science 223:1299: Jay, et al. (1984) J. Biol. Chem., 29:6311.

The assembled sequence can be cloned into any suitable vector or replicon and maintained there in a composition which is substantially free of vectors that do not contain the assembled sequence. This provides a reservoir of the assembled sequence, and segments or the entire sequence can be extracted from the reservoir by excising from DNA in the reservoir material with restriction enzymes or by PCR amplification. Numerous cloning vectors including commercially available cloning vectors are well known and readily available to those of skill in the art, and the selection of an appropriate cloning vector is a matter of choice (see, e.g., Sambrook, et al., incorporated herein by reference). The construction of vectors containing desired polynucleotide segments linked by appropriate polynucleotide sequences is accomplished by techniques similar to those used to construct the segments. These vectors may be constructed to contain additional DNA segments, such as bacterial origins of replication to make shuttle vectors (for shuttling between prokaryotic hosts and mammalian hosts), etc.

Procedures for construction and expression of polypeptides of defined sequence are well known in the art. A DNA sequence encoding polypeptides corresponding to pp32, or an analog thereof, can be synthesized chemically or prepared from the wild-type sequence by one of several approaches, including primer extension, linker insertion and PCR (see, e.g., Sambrook, et al.).

Mutants or variants can be prepared by these techniques having additions, deletions and substitutions in the wild-type pp32 sequence or portions thereof. It is preferable to test the mutants or variants to confirm that they are the desired sequence by sequence analysis and/or the assays described below. Mutant or variant polypeptides for testing may be prepared by placing the coding sequence for the polypeptides in a vector under the control of a promoter, so that the polynucleotide sequence is transcribed into RNA and translated into protein in a host cell transformed by this (expression) vector. The mutant protein may be produced by growing host cells transfected by an expression vector containing the coding sequence for the mutant under conditions whereby the polypeptides is expressed. The selection of the appropriate growth conditions is within the skill of the art.

The invention further includes polypeptide variants which exhibit the biological activity of inhibiting the association of pp32 with pRb. Such variants include deletions, insertions, inversions, repeats, and substitutions selected according to general rules known in the art so as have little effect on activity. For example, guidance concerning how to make phenotypically silent amino acid substitutions is provided in Bowie et al., Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

The assembled polynucleotide sequence can be cloned into any suitable vector or replicon and maintained there in a composition which is substantially free of vectors that do not contain the assembled sequence. This provides a reservoir of the assembled sequence, and segments or the entire sequence can be extracted from the reservoir by excising from polynucleotides in the reservoir material with restriction enzymes or by PCR amplification. Numerous cloning vectors are known to those of skill in the art, and the selection of an appropriate cloning vector is a matter of choice (see, e.g., Sambrook, et al., incorporated herein by reference). The construction of vectors containing desired DNA segments linked by appropriate DNA sequences is accomplished by techniques similar to those used to construct the segments. These vectors may be constructed to contain additional DNA segments, such as bacterial origins of replication to make shuttle vectors (for shuttling between prokaryotic hosts and mammalian hosts), etc.

Polypeptides

Polypeptides useful in the invention may be produced using methods that are well known to one of ordinary skill in the art. The polypeptides of the present invention are preferably provided in an isolated form, and preferably are substantially purified. A recombinantly produced version of a polypeptide, including the secreted polypeptide, can be substantially purified using techniques described herein or otherwise known in the art, such as, for example, by the one-step method described in Smith and Johnson, Gene 67:31-40 (1988). Polypeptides of the invention also can be purified from natural, synthetic or recombinant sources using techniques described herein or otherwise known in the art, such as, for example, antibodies of the invention raised against the polypeptides of the present invention in methods which are well known in the art.

Preferably, polynucleotides encoding pp32 polypeptides of interest should be subcloned into an expression vector, and the protein expressed by cells transformed with the vector should be tested for immunoreactivity with antibodies against the recombinant polypeptide(s) of this invention prepared as described below. Such subcloning is easily within the skill of the ordinary worker in the art in view of the present disclosure.

In one embodiment of the invention, the polypeptide coding region of the pp32 polynucleotide sequence of interest of this invention may be longer or shorter than the coding region of the disclosed sequence, so long as the recombinant pp32 polypeptide expressed by the polynucleotide sequence retains at least the activity of inhibiting the association of pp32 with pRb.

The preparation of selected clones which contain DNA sequences corresponding to all or part of the sequence of pp32 may be accomplished by those of ordinary skill in the art using conventional molecular biology techniques along with the information provided in this specification.

In one embodiment of the invention, recombinant polypeptides corresponding to portions of the pp32 protein may be obtained by transforming cells with an expression vector containing polynucleotide from a clone selected from an mammalian (preferably human) library as described herein. Suitable expression vector and host cell systems are well known to those of ordinary skill in the art, and are taught, for instance, in Sambrook, et al., 1989. The peptide may be obtained by growing the transformed cells in culture under conditions wherein the cloned polynucleotide is expressed.

In one embodiment of the invention, the polypeptide expressed by the clone may be longer or shorter than amino acids 67-120 of the human pp32 protein, so long as the polypeptides retain the ability to inhibit the association of pp32 with pRb. Depending on the expression vector chosen, the polypeptide(s) may be expressed as a fusion protein, a mature portion of pp32 or any fragment thereof which is secreted or retained intracellularly, or as an inclusion protein. The desired polypeptides can be recovered from the culture by well-known procedures, such as centrifugation, filtration, extraction, and the like, with or without cell rupture, depending on how the peptide was expressed. The crude aqueous solution or suspension may be enriched for the desired peptide by protein purification techniques well known to those skilled in the art. Preparation of the polypeptides may include biosynthesis of a protein including extraneous sequence which may be removed by post-culture processing.

Using the nucleotide sequences disclosed herein and the polypeptides expressed from them, antibodies can be obtained which have high binding affinity for pp32, fragments thereof, and other mutants or variants of wildtype pp32 polypeptides. Such antibodies, whether monoclonal or purified polyclonal antibodies can be used to specifically detect pp32, or fragment, mutants or variants thereof.

Techniques for preparing polypeptides, antibodies and nucleic acid probes for use in diagnostic assays, as well as diagnostic procedures suitable for detection of pp32 are described in U.S. Pat. Nos. 5,726,018 and 5,734,022, which are herein incorporated by reference, and these techniques may be applied to mutants or variants of pp32.

The polypeptide of the present invention can be attached to heterologous sequences to form chimeric or fusion proteins. Such chimeric and fusion proteins comprise a polypeptide peptide operatively linked to a heterologous protein having an amino acid sequence not substantially homologous to the polypeptide peptide. “Operatively linked” indicates that the polypeptide peptide and the heterologous protein are fused in-frame. The heterologous protein can be fused to the N-terminus or C-terminus of the polypeptide peptide. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired peptide can ultimately be separated from the fusion moiety. A chimeric or fusion protein can be produced by standard recombinant DNA techniques. For example, DNA fragments coding for the different protein sequences are ligated together in-frame in accordance with conventional techniques.

Further in accordance with this invention, the polypeptide may contain amino acids other than the 20 amino acids commonly referred to as the 20 naturally occurring amino acids and still retain the biological activity and substrate specificity of the polypeptide. Further, many amino acids, including the terminal amino acids, may be modified by natural processes, such as processing and other post-translational modifications, or by chemical modification techniques well known in the art. Known modifications include, but are not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutarnate, formylation, gamma carboxylation, glycosylation, GPI anchor formation, hydroxylation, iodination, lipid attachment, methylation, myristoylation, oxidation, proteolytic processing, phosphorylation, prenylation, racemization, selenoylation, sulfation, transfer-RNA mediated addition of amino acids to proteins such as arginylation, and ubiquitination. The modifications as described herein and still retain the biological activity and substrate specificity of the polypeptide.

Vectors

The invention also provides vectors containing the polynucleotides described herein. The term “vector” refers to a vehicle, preferably a nucleic acid molecule, which can transport the nucleic acid molecules. When the vector is a nucleic acid molecule, the nucleic acid molecules are covalently linked to the vector nucleic acid. The invention provides vectors for the maintenance (cloning vectors) or vectors for expression (expression vectors) of the nucleic acid molecules. The vectors can function in prokaryotic or eukaryotic cells or in both (shuttle vectors). With this aspect of the invention, the vector includes a plasmid, single or double stranded phage, a single or double stranded RNA or DNA viral vector, or artificial chromosome, such as a BAC, PAC, YAC, or MAC.

A variety of expression vectors can be used to express a polynucleotide. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, e.g. cosmids and phagemids. Plasmids suitable for this invention include but are not limited to pUC19, pBR322, pCMV, pSK Bluescript, pcDNA3, pcDNA3.1, pGEM, pGEX, pGST, pEGFP, and vectors that are otherwise commercially available and known to those of ordinary skill in the art. Appropriate cloning and expression vectors for prokaryotic and eukaryotic hosts are well known to those of ordinary skill in the art and many are described in Sambrook et al., Molecular Cloning: A Laboratory Manual. 2nd. ed., Cold Spring Harbor Laboratory Press, Cold Spring Harbor, N.Y., (1989).

The regulatory sequence to which the nucleic acid molecules described herein can be operably linked include promoters for directing mRNA transcription. These include, but are not limited to, the left promoter from bacteriophage λ, the lac, TRP, TAC, the early and late promoters from SV40, the CMV immediate early promoter, the adenovirus early and late promoters, retrovirus long-terminal repeats.

In addition to containing sites for transcription initiation and control, expression vectors can also contain initiation and termination codons, an origin of replication, polyadenylation signals, a ribosome binding site, repressor binding sites, and enhancers. The person of ordinary skill in the art would be aware of the numerous regulatory sequences that are useful in expression vectors. Such regulatory sequences are described, for example, in Sambrook et al., Molecular Cloning: A Laboratory Manual. 2nd. ed., Cold Spring Harbor Laboratory Press, Cold Spring Harbor, N.Y., (1989) which is incorporated by reference.

The regulatory sequence may provide constitutive expression in one or more host cells (i.e. tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The vectors of the present invention preferably contain one or more selectable markers which permit easy selection of transformed cells. A selectable marker is a gene the product of which provides for biocide or viral resistance, resistance to heavy metals, and prototrophy to auxotrophs. Examples of bacterial selectable markers are the dal genes from B. subtilis or B. licheniformis, or markers which confer antibiotic resistance such as ampicillin, kanamycin, chloramphenicol, zeomycin, or tetracycline resistance. A frequently used mammalian marker is the dihydrofolate reductase gene. Suitable markers for yeast host cells are ADE2, HIS3, LEU2, LYS2, MET3, TRP1, and URA3. Furthermore, selection may be accomplished by co-transformation where the selectable marker is on a separate vector as described in WO 91/17243 which is incorporated herein by reference.

A vector can be maintained in the host cell as an extrachromosomal element where it replicates and produces additional copies of the nucleic acid molecules. Alternatively, the vector may integrate into the host cell genome and produce additional copies of the nucleic acid molecules when the host cell replicates.

The polynucleotides can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

Gene Therapy Vectors

Gene Therapy vectors useful with the polynucleotides of the invention include, but are not limited to, retroviral, letiviral, and poxvirus vector systems. General teachings and methods regarding gene therapy vectors are known in the art, such as those disclosed in “Methods in Molecular Medicine: Gene Therapy Protocols”, Ed. Paul Robbins, Humana Press, 1997, the disclosure of which in herein incorporated by reference in its entirety.

In one embodiment of the invention, the gene therapy vector comprises a lentivirus based system. A non-primate lentivirus packageable nucleic acid is a nucleic acid having a functional virus packaging site from an ungulate lentivirus or feline immunodeficiency virus (FW) lentivirus. Nucleic acids having this packaging site which can be incorporated into a viral particle by viral components supplied in trans by a corresponding wild-type virus are packaged by the wild-type virus (or appropriate packaging components derived from a wild-type virus).

A packaging defect which blocks self packaging of a non-primate lentiviral vector nucleic acid is an inability of the nucleic acid to produce at least one viral protein necessary for packaging the vector nucleic acid into a viral particle in the context of a cell. For example, when Gag or Env proteins are not encoded by the lentiviral vector, the proteins must be supplied in trans before the vector nucleic acid can be packaged in the cell. The omission can be a deletion or mutation of a gene necessary for viral packaging from a viral clone, in the coding or non-coding (e.g., promoter) region of the relevant gene. The vector nucleic acid is trans-rescuable when it encodes a viral packaging site which is recognized be a non-primate lentiviral vector such as FIV.

A virus or vector “transduces” a cell when it transfers nucleic acid into the cell. A virus or vector is “infective” when it transduces a cell, replicates, and (without the benefit of any complementary virus or vector) spreads progeny vectors or viruses of the same type as the original transducing virus or vector to other cells in an organism or cell culture, wherein the progeny vectors or viruses have the same ability to reproduce and spread throughout the organism or cell culture. Thus, for example, a nucleic acid encoding an FIV particle is not infective if the nucleic acid cannot be packaged by the FIV particle (e.g., if the nucleic acid lacks an FIV packaging site), even though the nucleic acid can be used to transfect and transform a cell. Similarly, an FIV-packageable nucleic acid packaged by an FIV particle is not infective if it does not encode the FIV particle that it is packaged in, even though it may be used to transform and transfect a cell. Vectors which do not encode a complete set of viral packaging components (e.g., Gag and Env proteins) are “packaging deficient.” These vectors are “trans-rescuable” when the vectors are packaged by viral proteins supplied in trans in a packaging cell. If an FIV-packageable nucleic acid is used to transform a cell infected with FIV in a cell culture or organism infected with FIV, the FIV-packageable nucleic acid will be replicated and disseminated throughout the organism in concert with the infecting FIV virus. However, the FIV-packageable nucleic acid is not itself “infective”, because packaging functions are supplied by the infective FIV virus via trans complementation. Given the strategy for making the packaging plasmids and target packageable vector nucleic acids of the present invention, one of skill can construct a variety of clones containing functionally equivalent nucleic acids.

The non-primate lentiviruses include, but are not limited to, the ungulate lentiviruses, including visna/maedi virus, caprine arthritis encephalitis virus (CAEV), equine infectious anemia virus (EIAV), and bovine immunodeficiency virus (BIV). These lentiviruses only infect hoofed animals (ungulates) and generally only infect particular species of ungulates. The non-primate lentiviruses also include feline immunodeficiency virus (FIV) (see, Clements & Zink (1996) Clinical Microbiology Reviews 9, 100-117), which only infects felines. Numerous strains of FIV have been identified. Non-primate (e.g., feline and ungulate) lentiviruses may provide a safer alternative than primate lentiviral vectors, but their use is complicated by a relative lack of knowledge about their molecular properties, especially their adaptability to non-host animal cells. All lentiviruses display highly restricted tropisms (see, Clements & Zink (1996), supra, and Haase (1994) Annals of the New York Academy of Sciences 724, 75-86).

In another embodiment of the invention, the gene therapy vector comprises a poxvirus based system. Poxvirus includes but is not limited to vaccinia virus or avipox (e.g. canarypox or fowlpox), modified recombinant poxvirus-cytomegalovirus (CMV), human cytomegalovirus (HCMV) such as an attenuated recombinant, especially a NYVAC or ALVAC CMV or HCMV recombinant, Cowpox virus (Brighton red strain), fowlpoxvirus (FPV) and canarypoxvirus (CPV). Human cytomegalovirus (HCMV) is a member of the betaherpesviridae subfamily (family Herpesviridae).

Specifically, the recombinant poxviruses are constructed in two steps known in the art and analogous to the methods for creating synthetic recombinants of poxviruses such as the vaccinia virus and avipox virus described in U.S. Pat. Nos. 4,769,330, 4,772,848, 4,603,112, 5,100,587, and 5,179,993, the disclosures of which are incorporated herein by reference.

First, the DNA gene sequence to be inserted into the virus, particularly an open reading frame from a non-pox source, is placed into an E. coli plasmid construct into which DNA homologous to a section of DNA of the poxvirus has been inserted. Separately, the DNA gene sequence to be inserted is ligated to a promoter. The promoter-gene linkage is positioned in the plasmid construct so that the promoter-gene linkage is flanked on both ends by DNA homologous to a DNA sequence flanking a region of pox DNA containing a nonessential locus. The resulting plasmid construct is then amplified by growth within E. coli bacteria and isolated.

Second, the isolated plasmid containing the DNA gene sequence to be inserted is transfected into a cell culture, e.g. chick embryo fibroblasts, along with the poxvirus. Recombination between homologous pox DNA in the plasmid and the viral genome respectively gives a poxvirus modified by the presence, in a nonessential region of its genome, of foreign DNA sequences. The term “foreign” DNA designates exogenous DNA, particularly DNA from a non-pox source, that codes for gene products not ordinarily produced by the genome into which the exogenous DNA is placed.

A fine balance between the efficacy and the safety of a vaccinia virus-based recombinant vaccine candidate is extremely important. The recombinant virus used presents the immunogen(s) in a manner that elicits a protective immune response in the vaccinated animal but lacks any significant pathogenic properties. A number of vaccinia genes have been identified which are non-essential for growth of the virus in tissue culture and whose deletion or inactivation reduces virulence in a variety of animal systems.

Retrovirus (also known as retroviridae) is the taxonomic name for a family of RNA-containing viruses that have a reverse transcriptase. Their genome can be transcribed to DNA, which can be incorporated into a host cell's genome.

Retroviruses as vehicles for the delivery of genes into eukaryotic cells have several advantages: 1) gene transfer is relatively efficient; 2) stable integration into the host cell DNA is a natural part of the retroviral life cycle, and therefore the integrated provirus is passed on to all daughter cells, and continues to direct the non-lytic production of its encoded products; and 3) replication-defective vectors can be created by deletion of essential viral genes, which renders the vectors incapable of secondary infection.

Retrovirus that may be used in the instant invention include but are not limited to murine leukemia virus (MLV), murine sarcoma virus (MSV), and murine stem cell virus (MSCV).

Other gene therapy viral vectors which may be used in the instant invention include but are not limited to Adenoviruses, Adeno-associated viruses, and Herpes simplex viruses. Additional teachings regarding gene therapy vectors are known to those of ordinary skill in the art and are found for example in the following publications:

a) P. L. Sinn et al., “Gene Therapy Progress and Prospects: Development of Improved Lentiviral and Retroviral Vectors—Design, Biosafety, and Production”, Gene Therapy, Vol. 12, pp. 1089-1098 (2005);

b) L. De Laporte et al., “Design of Modular Non-Viral Gene Therapy Vectors”, Biomaterials, Vol. 27, pp. 947-954 (2006); and

c) P. Seth, “Vector-Mediated Cancer Gene Therapy”, Cancer Biology and Therapy, Vol. 4 (No. 5), pp. 512-517 (2005).

The disclosures of each of these publications is herein incorporated by reference in their entireties.

Host Cells

The vector containing the appropriate polynucleotide molecule can be introduced into an appropriate host cell for propagation or expression using well-known techniques. Host cells may be prokaryotic, including but not limited to bacterial cells, or eukaryotic, including but not limited to insect, fungal, mold, yeast, animal, and/or plant cells.

Bacterial host cells suitable for this invention may be gram positive or gram negative bacteria.

The gram positive bacteria suitable for this invention are preferably selected from the group of Bacillus species including but not limited to Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus circulans, Bacillus lautus, Bacillus megaterium, and Bacillus thuringiensis; or Streptomyces species such as Streptomyces lividans or Streptomyces murinus.

The gram negative bacteria suitable for this invention are preferably selected from the group of E. coli and Pseudomonas species. E. coli is particularly useful for this invention, in particular, the HB101, DH5α, JM101, JM109, and XL1-Blue strains.

Insect host cells suitable for this invention may be species preferably selected from the group of Sf9, Sf21, High-Five™ Cells, D.Mel-2 Cells, and Drosophila.

Fungi host cells suitable for this invention may be a species selected from the group of Achlya, Acremonium, Allomyces, Altemaria, Ascomycota, Aspergillus, Basidiomycota, Blastocladiella, Chytridiomycota, Coelomomyces, Emericella, Eumycota, Eupenicillium, Eurotium, Fusarium, Humicola, Mucor, Myceliophthora, Neurospora, Oomycota, Penicillium, Rhizopus, Saprolegniomycetous, Thielavia, Tolypocladium, Trichoderma, and Zygomycota.

Yeast host cells suitable for this invention may be species selected from the group of Blastomycetes, Bullera, Candida, Cryptococcaceae, Endomycetales, Filobasidiella, Filobasidium, Kluyveromyces, Leucosporidim, Lipomycoideae, Nadsonioideae, Pichia, Rhodosporidium. Saccharomyces, Saccharomycetaceae, Saccharomycoideae, Schizosaccharomyces, Schizosaccharomycoideae, Sorobolomyces, Spermophthoraceae, Sporidiobolus, Sporobolomycetaceae, and Yarrowia.

Animal host cells useful in this invention may be murine, human, bovine, porcine, ovine, canine, primate, and feline. Animal host cells useful for this invention including but not limited to A-375, A549, BAS8, BHK, bone marrow stem, C2C12, C6, Caco-2, CHO, CN1.4, COS, COS-7, D1-TNC1, D54, Daoy, DB-TRG-05, DU145, ES cells, fibroblasts, HEK 293, HeLa, Hep G2, Hepa 1-6, hepatocytes, HT-29, human astrocytes, IEC-18, Int407, Jurkat, keratinocytes, Keratinocytes (NIKS), L-929, M-24, macrophages, MCF-7, MG, MG-63, mouse germ cells, MRC-5, Neuro-2a, NIH 3T3, NT-2 cells, PC12, PC-3, primary cell lines, primary hepatocytes, RAW 264.7, SK-N-MC, THP-1, U-251, vascular endothelial, and Vero.

The recombinant host cells are prepared by introducing the vector constructs described herein into the cells by techniques readily available to the person of ordinary skill in the art. These include, but are not limited to calcium phosphate transfection, cationic lipid-mediated transfection, conjugation, DEAE-dextran-mediated transfection, electroporation, infection, lipofection, protoplast transformation, transduction, and other techniques such as those found in Sambrook, et al. Molecular Cloning: A Laboratory Manual. 2nd, ed, Cold Spring Harbor Laboratory, Cold Spring Harbor Laboratory Press, Cold Spring Harbor, N.Y., 1989, which is incorporated by reference.

Where the peptide is not secreted into the medium, which is typically the case with polypeptides, the protein can be isolated from the host cell by standard disruption procedures, including freeze thaw, sonication, mechanical disruption, use of lysing agents and the like. The peptide can then be recovered and purified by purification methods known in the art including but not limited to acid extraction, affinity chromatography, ammonium sulfate precipitation, anion exchange chromatography, cationic exchange chromatography, high performance liquid chromatography (HLPC), hydrophobic-interaction chromatography, hydroxylapatite chromatography, lectin chromatography, and phosphocellulose chromatography.

Screening Assays

The invention also includes screening assays designed to identify those candidate agents capable of inhibiting the association of pp32 with pRb. In one embodiment of the invention, the assay comprises mixing a candidate agent with pp32 and pRb proteins, and measuring the binding of pp32 to pRb in the presence of the candidate agent.

It is apparent to one of ordinary skill in the art that numerous methods of detecting the association of pp32 and pRb in the presence of a candidate agent are available. In a preferred embodiment of the invention, the association of pp32 to pRb in the presence of the candidate agent is detected using co-immunoprecipitation, followed by gel electrophoresis and Western blotting, as exemplified in the following Examples section of this specification. As contemplated herein, pp32 and pRb do not need to be supplied as pure components, but may be provided in mixtures or unpurified forms of compositions, such as cell lysates.

Administration

In one embodiment of the invention, one or more of the agents described herein are administered to a subject in conjunction with cationic cell penetrating peptides. Teachings related to cationic cell penetrating peptides are known to those of ordinary skill in the art and can be found for example in the following publications:

a) T. Shiraishi et al., “Photochemically Enhanced Cellular Delivery of Cell Penetrating Peptide-PNA Conjugates”, FEBS Letters, Vol. 580 (no. 5), pp. 1451-1456 (2006); and

b) I. Massoudi et al., “Evaluation of Cell Penetrating Peptides Fused to Elastin-like Polypeptide for Drug Delivery”, Journal of Controlled Release, Vol. 108 (no. 2-3), pp. 396-408 (2005).

The disclosures of each of these publications is herein incorporated by reference in their entireties.

The inhibitory agents described herein can be co-administered with one or more chemotherapeutic agents. Chemotherapeutic agents that may be administered with the therapeutics of the invention include, but are not limited to alkylating agents such as nitrogen mustards (for example, Mechlorethamine, cyclophosphamide, Cyclophosphamide Ifosfamide, Melphalan (L-sarcolysin), and Chlorambucil), ethylenimines and methylmelamines (for example, Hexamethylmelamine and Thiotepa), alkyl sulfonates (for example, Busulfan), nitrosoureas (for example, Carmustine (BCNU), Lomustine (CCNU), Semustine (methyl-CCNU), and Streptozocin (streptozotocin)), triazenes (for example, Dacarbazine (DTIC; dimethyltriazenoimidazolecarboxamide)), folic acid analogs (for example, Methotrexate (amethopterin)), pyrimidine analogs (for example, Fluorouacil (5-fluorouracil; 5-FU), Floxuridine (fluorodeoxyuridine; FudR), and Cytarabine (cytosine arabinoside)), purine analogs and related inhibitors (for example, Mercaptopurine (6-mercaptopurine; 6-MP), Thioguanine (6-thioguanine; TG), and Pentostatin (2′-deoxycoformycin)), vinca alkaloids (for example, Vinblastine (VLB, vinblastine sulfate)) and Vincristine (vincristine sulfate)), epipodophyllotoxins (for example, Etoposide and Teniposide), antibiotics (for example, Dactinomycin (actinomycin D), Daunorubicin (daunomycin; rubidomycin), Doxorubicin, Bleomycin, Plicamycin (mithramycin), and Mitomycin (mitomycin C), enzymes (for example, L-Asparaginase), biological response modifiers (for example, Interferon-alpha and interferon-alpha-2b), platinum coordination compounds (for example, Cisplatin (cis-DDP) and Carboplatin), anthracenedione (Mitoxantrone), substituted ureas (for example, Hydroxyurea), methylhydrazine derivatives (for example, Procarbazine (N-methylhydrazine; M1H), adrenocorticosteroids (for example, Prednisone), progestins (for example, Hydroxyprogesterone caproate, Medroxyprogesterone, Medroxyprogesterone acetate, and Megestrol acetate), estrogens (for example, Diethylstilbestrol (DES), Diethylstilbestrol diphosphate, Estradiol, and Ethinyl estradiol), antiestrogens (for example, Tamoxifen), androgens (Testosterone proprionate, and Fluoxymesterone), antiandrogens (for example, Flutamide), gonadotropin-releasing hormone analogs (for example, Leuprolide), other hormones and hormone analogs (for example, methyltestosterone, estramustine, estramustine phosphate sodium, chlorotrianisene, and testrolactone), and others (for example, dicarbazine, glutamic acid, and mitotane).

In another embodiment, the compostions of the invention are administered in combination with one or more anti-angiogenic agents.

In a preferred embodiment of the invention, the compositions of the invention are administered in combination with one or more of the following chemotherapeutic agents: Mitoxantrone; Prednisone; Paclitaxel; Docetaxel; Estramustine; and Adriamycin.

A “pharmaceutical composition” refers to a chemical or biological composition suitable for administration to a mammal. Such compositions may be specifically formulated for administration via one or more of a number of routes, including but not limited to buccal, epicutaneous, epidural, inhalation, intraarterial, intracardial, intracerebroventricular, intradermal, intramuscular, intranasal, intraocular, intraperitoneal, intraspinal, intrathecal, intravenous, oral, parenteral, rectally via an enema or suppository, subcutaneous, subdermal, sublingual, transdermal, and transmucosal. In addition, administration can by means of injection, tablet, pill, powder, liquid, gel, capsule, porous pouch, drops, patch, foams, or other means of administration.

A “pharmaceutical excipient” or a “pharmaceutically acceptable excipient” is a carrier, usually a liquid, in which an active therapeutic agent is formulated. The excipient generally does not provide any pharmacological activity to the formulation, though it may provide chemical and/or biological stability, and release characteristics. Exemplary formulations can be found, for example, in Remington's Pharmaceutical Sciences, 19^{th} Ed., Grennaro, A., Ed., 1995 which is incorporated by reference.

As used herein “pharmaceutically acceptable carrier” or “excipient” includes any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the alkaline polypeptide can be formulated in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

The preferred forms of administration in the present invention are oral forms know in the art of pharmaceutics. The pharmaceutical compositions of the present invention may be orally administered as a capsule (hard or soft), tablet (film coated, enteric coated or uncoated), powder or granules (coated or uncoated) or liquid (solution or suspension). The formulations may be conveniently prepared by any of the methods well-known in the art. The pharmaceutical compositions of the present invention may include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients, e.g., solid and liquid diluent, such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The above description of various illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other purposes, other than the examples described above.

These and other changes can be made to the invention in light of the above detailed description. In general, in the following claims, the terms used should not be construed to limit the invention to the specific embodiments disclosed in the specification and the claims. Accordingly, the invention is not limited by the disclosure, but instead the scope of the invention is to be determined entirely by the following claims.

The invention may be practiced in ways other than those particularly described in the foregoing description and examples. Numerous modifications and variations of the invention are possible in light of the above teachings and, therefore, are within the scope of the appended claims.

Certain teachings related to pp32 and hyperphosphorylated Retinoblastoma (pRb) were disclosed in U.S. provisional patent application No. 60/675,565, filed Apr. 28, 2005, as well as in the publication by O. Adegbola and G. Pasternack entitled “Phosphorylated Retinoblastoma Protein Complexes with pp32 and Inhibits pp32-mediated Apoptosis”, published in the Journal of Biological Chemistry, Vol. 280, No. 16, pp. 15497-15502 (2005), the disclosures of each of which are herein incorporated by reference in their entireties.

The entire disclosure of each document cited (including patents, patent applications, journal articles, abstracts, manuals, books, or other disclosures) in the Background of the Invention, Detailed Description, and Examples is herein incorporated by reference in their entireties.

### EXAMPLES

### Example 1

### Screening for pp32 Interactions by Immunoprecipitation and Identification of Rb as a Protein Interacting with pp32

Construction of vectors—All polymerase chain reaction (PCR) reagents were purchased from Qiagen. Primers were designed using the Stanford primer program (http://genome-www2.stanford.edu/cgi-bin/SGD/web-primer). All pp32 constructs utilized a common upstream primer, and all downstream primers lacked a stop codon to facilitate COOH-terminal V5 epitope tagging. Following amplification from pp32 plasmids, products were cloned into the expression vector pcDNA3.1/V5-His Topo TA (Invitrogen) according to the manufacturer's instructions. Both amplicons were Nhe1 digested and ligated, and pp32 upstream and downstream primers were used to amplify the ligation products. The PCR products were then cloned into pcDNA3.1/V5-His Topo TA (Invitrogen). All constructs were verified by sequencing. Cells were maintained in Dulbelcco's modified Eagle's medium (DMEM, Invitrogen) with 10% fetal bovine serum (GIBCO) and 1% penicillin/streptomycin (P/S, GIBCO). Cells were passaged 2-3 times/week. All DNA transfections were carried out using Fugene 6 (Roche) as described by the manufacturer.

Immunoprecipitation and Immunoblotting-1×10⁶ HEK 293 cells were seeded onto a T-150 flask. 24 hours later, cells were transfected with 15 μg of the indicated plasmids. 48 hrs post transfection, cells were harvested, washed twice with cold PBS, and lysed with MPER (Pierce) containing 1× protease inhibitor cocktail (HALT, Pierce). Lysates were centrifuged at 4° C. for 30 minutes at 16,000 g to remove particulate material. The supernatant was precleared for 2 hours with protein A-agarose (Roche). The pre-cleared cell lysates were mixed with the indicated antibodies and protein A- or protein G-agarose (Roche) and incubated at 4° C. overnight. The next day, the reaction mixture was washed three times with cold PBS, boiled for 3 minutes and eluted in 2×SDS buffer. The eluted materials were subsequently analyzed by immunoblotting with the indicated antibodies.

Antibodies—The following antibodies were used for this study: anti-pp32; anti-V5 (Invitrogen); anti-E2F (KH95, BD Biosciences); anti-Rb G3-245, anti-Rb G99-549, anti-Rb G99-2005 (BD Biosciences); anti-Rb C-15 (Santa Cruz); and polyclonal anti-RB 851 (gift from Erik Knudsen). The following anti-phospho-Rb antibodies were used: anti-S795 (Cell Signalling); anti-T249/252, anti-T356, anti-S612, anti-S780, anti-S807/811, anti-T821, and anti-T826, (Biosource International).

Proteins were separated in NuPAGE 10% Bis-Tris gel (Invitrogen) and electroblotted onto PVDF membranes (Invitrogen). Immunoblot analysis used indicated specific antibodies and enhanced chemoluminescence (ECL)-based detection (Amersham). Where indicated, blots were stripped with Restore western blot stripping buffer (Pierce) as per the manufacturer's instructions.

FIG. 1A shows that Rb co-immunoprecipitates with V5-tagged pp32. HEK 293 cells were transfected with pp32V5. Equal amounts of cell extracts were precipitated with anti-V5 or a control (NSE) antibody and the presence of Rb in the immunoprecipitates was visualized by western blot analysis using anti-Rb (G3-245) antibody. FIG. 1B shows that V5-tagged pp32 co-immunoprecipitates with Rb. HEK 293 cells were transfected with pp32V5 and lacZV5 as indicated. Equal amounts of cell extracts were precipitated with anti-Rb (G99-2005) antibody and the presence of V5 in immunoprecipitates was visualized by western blot analysis using anti-V5 antibody. FIG. 1C shows that endogenous pp32 co-immunoprecipitates with endogenous Rb. Equal amounts of HeLa cell extracts were precipitated with anti-Rb (G99-2005) or a control (PCNA) antibody and the presence of pp32 in immunoprecipitates was visualized by western blot analysis using anti-pp32 antibody. Identical results were obtained with HEK 293 and K562 cells.

When V5-epitope tagged pp32 is expressed by transient transfection of mammalian cells an interaction between endogenous Rb and pp32V5 is detected by western blot analysis following immunoprecipitation with either anti-V5 (FIG. 1A) or anti-Rb antibody (FIG. 1B). The interaction between Rb and pp32 can also be demonstrated in untransfected mammalian cells, including HeLa cells (FIG. 1C), suggesting that it is physiologically relevant.

### Example 2

### pp32 Interacts with Rb Via an LRR Motif

pp32 is made up of a nuclear localization signal (NLS), an acidic region and a leucine rich repeat (LRR) region that contains the NLS. Suppression of transformation and INHAT activity map to amino acids 150 to 174, slightly N-terminal to the acidic region. LRRs generally mediate protein-protein interactions (Ohsumi, T., Ichimura, T., Sugano, H., Omata, S., Isobe, T., and Kuwano, R. (1993) Biochem. J. 294, 465-472), and the LRR of pp32 mediates its nucleocytoplasmic shuttling via binding to CRM1 (Brennan, C. M., Gallouzi, I. E., and Steitz, J. A. (2000) J. Cell Biol. 151, 1-14).

The Rb-binding region of pp32 was mapped using V5-epitope tagged constructs lacking the acidic region, the LRR or both (FIG. 1D). Vectors were constructed as described for Example 1. pp32 truncation constructs were generated via PCR amplification of desired pp32 sequences. The upstream primer of bases 360-747 and the downstream primer of bases 1-201 had Nhe1 sites at their 5′ ends. E2 μl and pRb plasmids were kind gifts from Fikret Sahin (Johns Hopkins) and Robert Weinberg (MIT) respectively.

A schematic diagram of pp32 mutants is shown in FIG. 1D. Nucleic acid number of domain boundaries is indicated. All contain a COOH-terminal V5 epitope tag. FIG. 1E show that Rb interacts with the LRR of pp32. HEK 293 cells were transfected with the indicated V5 epitope tagged mutants. Culture and transfection of cells were carried out as described for Example 1. In both panels of FIG. 1E, the unlabeled lane on the left represents molecular weight markers of 20, 30, and 40 kDa. Upper panel of FIG. 1E shows equal amounts of cell extracts which were precipitated with anti-Rb (G99-2005) antibody and the presence of V5 in immunoprecipitates was visualized by western blot analysis using anti-V5 antibody. The arrow indicates the position of immunoglobulin light chain. In the lower panel, cell extracts were subjected to anti-V5 western blot analysis to confirm expression of the indicated V5 epitope tagged mutants. FIG. 1F shows a replicate of the experiment shown in FIG. 1E restricted to pp32V5 and pp32V5-201 at a higher level of expression.

While deletion of the acidic region had no effect, deletion of nucleotides 201-360, encoding amino acids 67-120 in the LRR of pp32, abolished Rb binding (FIG. 1E). Because the expression of pp32V5-201 appeared to be low in the lysate shown in FIG. 1E, the experiment was repeated with a higher expression level (FIG. 1F) yielding the identical result.

### Example 3

### Which Form of Rb Interacts with pp32

As Rb functions are regulated by phosphorylation, the next experiment determined which form of Rb (hypo- or hyper-phosphorylated) interacted with pp32. Transfection and immunoprecipitation were performed as described above. In FIG. 2A, HEK 293 cells were transfected with pp32V5 or sham transfected. Equal amounts of transfected cell extracts were precipitated with anti-V5 or a control (NSE) antibody as indicated. Equal amounts of sham transfected cell extracts were precipitated with total Rb (C-15) or control (AChE) antibody as indicated. The presence of hypophosphorylated Rb in the immunoprecipitates was analysed by immunoblotting using an antibody specific for hypophosphorylated Rb (G99-549). In FIG. 2B. the blot in FIG. 2A was stripped and re-probed with total Rb (G3-245) antibody. V5 epitope tagged pp32 was transiently expressed in HEK 293 cells and anti-V5 antibody was used to immunoprecipitate pp32V5. pp32 co-immunoprecipitates with hyperphosphorylated Rb since the V5 immunoprecipitates did not contain any hypophosphorylated Rb (FIGS. 2A and B).

### Example 4

### pp32 Binds Preferentially to Rb Phosphorylated on T⁸²⁶

Although not all Rb phosphorylation sites in vivo have been identified, Rb has at least 16 predicted cdk phosphorylation sites (Lees, J. A., Buchkovich, K. J., Marshak, D. R., Anderson, C. W., and Harlow, E. (1991) EMBO J. 10, 4279-4290). Differentially phosphorylated forms of Rb appear to exist in cells (DeCaprio, J. A., Ludlow, J. W., Lynch, D., Furukawa, Y., Griffin, J., Piwnica-Worms, H., Huang, C. M., and Livingston, D. M. (1989) Cell 58, 1085-1095) and there is evidence that differential phosphorylation of Rb may regulate its functions. Phosphorylation of S⁸⁰⁷ and S⁸¹¹ regulate binding of Rb to c-Abl, while phosphorylation of T⁸²¹ and T⁸²⁶ regulate binding to LXCXE proteins (Knudsen, E. S., and Wang, J. Y. J (1996) J. Biol. Chem. 271, 8313-8320). To determine the specific phosphorylated form of Rb that binds to pp32, various phosphospecific Rb antibodies were used to probe an anti-V5 immunoprecipitate of pp32V5.

Cell culture and transfections were performed as in Example 3. Immunoprecipitation was performed as described in Example 1. HEK 293 cells were transfected with pp32V5, LacZV5 or sham transfected as indicated. Equal amounts of pp32V5 and LacZV5 transfected cell extracts were precipitated with anti-V5. As a positive control, sham transfected cell extracts were precipitated with antibody to total Rb (G3-245); this control, which precipitates considerably more Rb, is designated “Sham” on the figure. The presence of specific phosphorylated forms of Rb in the immunoprecipitates was analysed by immunoblotting using the indicated anti-phospho-Rb antibodies. Immunoprecipitates specifically reacted with an anti-phosphoT⁸²⁶ Rb antibody (FIG. 2C).

### Example 5

### Confirmation that pp32 Binds to phosphoT⁸²⁶ Rb

To further confirm that Rb phosphorylation at T⁸²⁶ is necessary for pp32 binding, Rb large pocket constructs WT-LP and PSM2T-LP which have previously been described (Knudsen, et al., 1996) were used in experiments performed according to the procedures discussed above. Rb large pocket constructs WT-LP and PSM2T-LP were kind gifts from Erik Knudsen (University of Cincinnati). WT-LP encodes the wild-type large pocket fragment of RB (amino acids 379-928) and PSM2T-LP is a double T⁸²¹ A/T⁸²⁶A large pocket mutant.

HEK 293 cells were cotransfected with the pp32V5 and control, WT-LP or PSM2T-LP as indicated in FIG. 2D. Equal amounts of cell extracts were precipitated with anti-V5 antibody. pp32 co-immunoprecipitated with WT-LP but not PSM2T-LP, suggesting that pp32-Rb interaction requires Rb phosphorylation at T⁸²⁶, as shown in FIG. 2D. Upper panel shows the presence of WT-LP or PSM2T-LP in immunoprecipitates probed by western blot analysis using anti-Rb antibody (851). The right-hand arrow indicates the position of the Rb large pocket fragment. The lower band present in all three lanes is immunoglobulin heavy chain, which serves as a loading control. In the middle panel, anti-V5 immunoprecipitates were subjected to western blot analysis with anti-V5 antibody to confirm pp32V5 immunoprecipitation. Lower panel shows cell extracts which were subjected to western blotting with anti-Rb antibody (851) to confirm expression of WT-LP and PSM2T-LP.

### Example 6

### pp32 Increases E2F1-Mediated Transcriptional Activity

As a tumor suppressor, Rb inhibits proliferation by repressing E2 μl mediated transcription when hypophosphorylated. Hyperphosphorylation of Rb relieves E2 μl repression and allows cell cycle progression to occur (Nevins, J. R., (2001) Hum. Mol. Genet. 10, 699-703). Because hyperphosphorylated Rb is unable to repress E2 μl mediated transcription, an E2F-luciferase reporter plasmid was used to investigate whether pp32 could increase E2F1-mediated transcriptional activity.

Reporter assays—For reporter assays, NIH 3T3 cells were transfected with 1 μg of E2F-TA-LUC (Clontech), 0.5 μg of E2F1, 0.5 μg of pRb, and 1 μg of pp32V5 or pp32Δ201-360V5 expression plasmids as indicated. In all the samples, 50 ng of the reporter vector pRL-TK (Promega) was included for normalization of the transfection efficiency. Total transfected DNA was kept constant at 3 μg with pcDNA 3.1 when necessary. 24 hours after transfections, cells were lysed and assayed for luciferase activity using the Dual Luciferase kit (Promega) as per the manufacturer's protocol.

Statistical analysis of the reporter data was carried out by one-way ANOVA followed by a Tukey multiple comparison post-test to compare individual pairs of data sets. The analysis was performed using GraphPad Prism software, v. 4.00 (www.graphpad.com).

Overexpression of pp32 consistently resulted in 3-5-fold increased transactivation of the E2F-luciferase promoter in the presence of excess E2 μl (p<0.001 for E2 μl+pp32 vs. control, FIG. 3). In the experiment show in FIG. 3, NIH 3T3 cells were transiently transfected with E2F-luciferase reporter vector (pE2F-TA-Luc) and, where indicated, E2F1, pRb, pp32V5 or pp32D201-360V5 expression vectors. Data are presented as the mean±SEM from three independent experiments performed in duplicate. This increased transcriptional activation could be decreased by overexpression of Rb (p<0.001 for E2F1+pp32 vs. E2F1+pRB+pp32) and completely abolished by disruption of the interaction between Rb and pp32 by deletion of amino acids 67-120 (p<0.001, E2F1+pp32 vs. E2F1+pp32Δ201-360). Cells transfected with the reporter plus pp32 alone, E2F1 alone, E2F1+Rb, E2F1+pRB+pp32, E2F1+pp32Δ201-360, or E2F1+pRB+pp32Δ201-360 did not differ significantly from the control or from one another (p>0.05).

These results suggest that pp32 is able to sequester hyperphosphorylated Rb and thereby increase free E2F1. At the doses used in these assays, pp32 did not increase E2F-luciferase transactivation in the absence of E2F1 overexpression. It is probable that because there was enough endogenous Rb to bind E2F1 and pp32, the sequestration of Rb by pp32 was only unmasked when excess E2F1 was added to the system. The fact that pp32 has an acidic domain found in transcriptional activators raised the possibility that pp32 directly interacts with E2F1 to increase E2F1-mediated transcription. pp32 was transiently overexpressed in HEK 293 cells and cell lysates were immunoprecipitated with E2 μl and Rb antibodies. While pp32 was detected in Rb immunoprecipitates, it was absent in E2F1 immunoprecipitates (data not shown). These results rule out both direct and indirect interactions between pp32 and E2F1.

### Example 7

### Association Between Rb and pp32 Correlates with Inhibition of pp32 Apoptotic Activity

As pp32 is pro-apoptotic, while Rb is anti-apoptotic, this experiment is directed to investigation of the effect of Rb on the apoptotic function of pp32.

Apoptosis assays—In mammalian cells, transient overexpression of pp32 resulted in increased apoptosis as assessed by Hoechst staining (FIG. 4A). 1.5×10⁵ HeLa cells were seeded in 6 well plates overnight. 24 hours later, they were transfected with 1 μg of DNA containing the indicated plasmids, and 1 μg of vector control plasmid where necessary for a total of 2 μg DNA. 48 hours post transfection, cells were fixed with ice cold 100% methanol at −20° C. for 15 min. After fixation, cells were stained with 10 μg ml⁻¹ Hoechst 33342 (Molecular Probes) for 10 min at 37° C. Samples were mounted with mounting medium containing Prolong antifade reagent (Molecular Probes). Apoptotic cells were identified and counted using a Nikon microscope equipped with an epi-illuminator and appropriate filters. The percentages of apoptotic cells were determined from 300 cells counted in 3 each of 3 independent experiments.

pp32 induced apoptosis is abrogated by Rb in mammalian cells, as shown in FIG. 4A. HeLa cells were transfected with expression plasmids encoding the indicated proteins. Nuclei were stained with Hoechst stain and examined by immunofluorescence microscopy for characteristics of apoptosis (membrane blebbing, chromatin condensation and pyknosis). Cell death was quantified in HeLa cells transfected with the indicated expression constructs. The data (mean±SEM) are the percentage of nuclei counted with apoptotic morphology (n equals at least three experiments). The apoptotic effect of pp32 was abolished by coexpression of Rb.

### Example 8

### Apoptotic Activity of pp32 in Mammalian Cells is Inhibited by Rb

To further evaluate the effect of Rb on the pro-apoptotic activity of pp32 in mammalian cells, colony formation assays were performed.

Colony formation assays—pp32 overexpression results in a decrease in colony formation compared to vector control (FIG. 4B). 1.5×10⁵ NIH 3T3 or HeLa cells, as indicated, were seeded in 6 well plates overnight. 24 hours later, they were transfected with 1 μg of DNA containing the indicated plasmids (see above). Total transfected DNA was kept constant at 2 μg with pcDNA 3.1 when necessary. 48 hours after transfection, cells were split into a 100-mm dish containing DMEM/10% P/S supplemented with 1000-500 μg/ml G418 (Gibco). The cultures were fed every 3-4 days. After 2 weeks, the cells were fixed with 95% ethanol, stained with 0.5% crystal violet in 95% ethanol, plates photographed and colonies counted.

Cotransfection of Rb with pp32 abrogated the pp32 mediated decrease in colony formation (FIG. 4B). Duplicate plates of HeLa cells were transfected with control, pp32, or pRb expression plasmids as indicated and subjected to colony formation assay. Plates were stained with methylene blue and total number of G418 resistant colonies were counted after 14 days of selection. A representative experiment is shown. The bar graph (mean±SEM) shows the percentage change in colony formation efficiency with the colony counts normalized against the vector only control (n equals at least three experiments in duplicate). The pp32Δ201-360 construct yielded greatly diminished levels of apoptosis, which precluded demonstration that it was insensitive to the addition of Rb; this experiment would have provided more direct evidence that the apoptotic effects of pp32 are inhibited by Rb rather than by another mechanism.

### Example 9

### Apoptotic Activity of pp32 in Mammalian Cells is Inhibited by Rb

NIH 3T3 is a classic cell system for testing various transformation agents. Overexpression of v-H-Ras protein in NIH 3T3 cells results in cellular transformation and accelerated cell cycle progression associated with an increased level of cyclin D, which increases hyperphosphorylated Rb levels (Liu, J. J., Chao, J. R., Jiang, M. C., Ng, S. Y., Yen, J. J. and Yang-Yen, H. F. (1995) Mol. Cell. Bio. 15, 3654-3663). To examine the effect of the pp32-Rb interaction on mitogenesis, activated H-ras, pp32 and pRb were transfected into NIH 3T3 cells as described in Example 6.

Duplicate plates of NIH 3T3 cells were transfected with ras, pp32, or pRb expression plasmids as indicated above and subjected to colony formation assay. Plates were stained with methylene blue and photographed after 14 days of G418 selection. Coexpression of ras and pp32 resulted in a slight decrease in colony formation while coexpression of ras, pp32 and Rb resulted in markedly increased colony formation compared to the ras only control (FIG. 4C).

### Example 10

### Rb Associates with pp32, But not with Other Members of the ANP32 Family

While pp32 inhibits transformation, pp32r1 (ANP32C) and pp32r2 (ANP32D), both highly homologous to pp32 at the protein level (87.7% and 89.3% respectively) are tumorigenic (Kadkol, S. S., Brody, J. R., Pevsner, J., Bai, J., and Pasternack, G. R. (1999) Nature Med. 5, 275-279). Therefore, the possibility of an interaction between these family members and Rb was explored. pp32r1 and r2 were V5-epitope tagged and overexpressed in HEK 293 cells. HEK 293 cells were transfected with pp32V5, pp32r1V5, pp32r2V5 or LacZV5 expression vectors as indicated. Equal amounts of cell extracts were precipitated with anti-Rb (G99-2005) antibody and analysed by western blotting with anti-V5 antibody. Equal amounts of cell lysates were immunoprecipitated with anti-Rb and immunoblotted with anti-V5. Surprisingly, pp32r1 and pp32r2 did not interact with Rb (FIG. 5A) despite high conservation within amino acids 67-120 of the LRR region (88.9% and 90.7% identical respectively), suggesting that the interaction between Rb and pp32 is highly specific.

### REFERENCES

1. Nevins, J. R., (2001)Hum. Mol. Genet. 10, 699-703
2. Sherr, C. J., (1996) Science 274, 1672-1677
3. Almasan, A., Yin, Y., Kelly, R. E., Lee, E. Y., Bradley, A., Li, W., Bertino, J. R., and Wahl, G. M. (1995) Proc. Natl. Acad. Sci. U SA 92, 5436-5440
4. Berry, D. E., Lu, Y., Schmidt, B., Fallon, P. G., O'Connell, C., Hu, S. X., Xu, H. J., and Blanck, G. (1996) Oncogene 12, 1809-1819
5. Lee, E. Y., Chang, C., Hu, N., Wang, Y. C., Lai, C. C., Herrup, K., Lee, W. H., and Bradley, A. (1992) Nature 359, 288-294
6. Jacks, T., Fazeli, A., Schmitt, E. M., Bronson, R. T., Goodell, M. A., and Weinberg, R. A. (1992) Nature 359, 295-300
7. Macleod, K. F., Hu, Y., and Jacks, T. (1996) EMBO J. 15, 6178-6188
8. Sage, J., Mulligan, G. J., Attardi, L. D., Miller, A., Chen, S., Williams, B., Theodorou, E., and Jacks, T. (2000) Genes Dev. 14, 3037-3050
9. Dannenberg, J. H., van. Rossum, A., Schuijff, L., and te Riele, H. (2000) Genes Dev. 14, 3051-3064
10. Wang, J., Guo, K., Wills, K. N., and Walsh, K. (1997) Cancer Research 57, 351-354
11. Harbour, J. W., and Dean, D. C. (2000) Nat. Cell Biol. 2, E65-67.
12. Shackney, S. E., and Shankey, T. E. (1999) Cytometry 35, 97-116
13. Ma, D., Zhou, P., and Harbour, J. W. (2003) J. Biol. Chem. 278, 19358-19366
14. Berndt, N., Dohadwala, M., and Liu, C. W. (1997) Curr Biol. 7, 375-386
15. Wang, R. H., Liu, C. W., Avramis, V. I., and Berndt, N. (2001) Oncogene 20, 6111-6122
16. Morana, S. J., Wolf, C. M., Li, J., Reynolds, J. E., Brown, M. K., and Eastman, A. (1996) J. Biol. Chem. 271, 18263-18271
17. Dou, Q. P., An, B., and Will, P. L. (1995) Proc. Natl. Acad. Sci. U.S.A. 92, 9019-9023
18. Joseph, L. F., Ezhevsky, S., and Scott, D. W. (1995) Cell Growth Differ. 6, 51-57
19. Wang, H., Grand, R. J., Milner, A. E., Armitage, R. J., Gordon, J., and Gregory, C. D. (1996) Oncogene 13, 373-379
20. Wallace, M., Coates, P. J., Wright, E. G., and Ball, K. L. (2001) Oncogene 20, 3597-3608
21. Rassidakis, G. Z., Lai, R., Herling, M., Cromwell, C., Schmitt-Graeff, A., and Medeiros, L. J. (2004) Am. J. Pathol. 164, 2259-2267
22. Tsai, K. Y., Hu, Y., Macleod, K. F., Crowley, D., Yamasaki, L., and Jacks, T. (1998) Mol. Cell. 3, 293-304
23. Chau, B. N., Borges, H. L., Chen, T. T., Masselli, A., Hunton, I. C., and Wang, J. Y. (2002) Nat. Cell Biol. 4, 757-765
24. Chau, B. N., and Wang, J. Y. (2003) Nat. Rev. Cancer 3, 130-138
25. Morris, E. J., and Dyson, N. J. (2001) Adv. Cancer Res. 82, 1-54
26. Walensky, L. D., Coffey, D., Chen, T. H., Wu, T. C., and Pasternack, G. R. (1993) Cancer Res. 53, 4720-4726
27. Malek, S. N., Katumuluwa, A. I., and Pasternack, G. R. (1990) J. Biol. Chem. 265, 13400-13409
28. Matsuoka, K., Taoka, M., Satozawa, N., Nakayama, H., Ichimura, T., Takahashi, N., Yamakuni, T., Song, S. Y., and Isobe, T. (1994) Proc. Natl. Acad. Sci. USA 91, 9670-9674
29. Fan, Z., Beresford, P. J., Oh, D. Y., Zhang, D., and Lieberman, J. (2003) Cell 112, 659-672
30. Jiang, X., Kim, H. E., Shu, H., Zhao, Y., Zhang, H., Kofron, J., Donnelly, J., Burns, D., Ng, S. C., Rosenberg, S., and Wang, X. (2003) Science 299, 223-226
31. Brody, J. R., Kadkol, S., Mahmoud, M. A., Rebel, J. M. and Pasternack G. R. (1999) J. Biol. Chem. 274, 20053-20055
32. Chen, T. H., Brody, J. R., Romantsev, F. E., Yu, J. G., Kayler, A. E., Voneiff, E., Kuhajda, F. P., and Pasternack G. R. (1996) Mol. Biol. Cell 7, 2045-2056
33. Li, M., Makkinje, A., and Damuni, Z. (1996) Biochemistry 35, 6998-7002.
34. Brennan, C. M., Gallouzi, I. E., and Steitz, J. A. (2000) J. Cell Biol. 151, 1-14
35. Seo, S. B., McNamara, P., Heo, S., Turner, A., Lane, W. S., and Chakravarti, D. (2001) Cell 104, 119-130
36. Bai, J., Brody, J. R., Kadkol, S. S., and Pasternack, G. R. (2001) Oncogene 20, 2153-2160
37. Brody, J. R., Kadkol, S. S., Hauer, M. C., Rajaii, F., Lee, J., and Pasternack, G. R. (2004) Am. J. Pathol. 164, 273-283
38. Kadkol, S. S., Brody, J. R., Epstein, J. I., Kuhajda, F. P., and Pasternack, G. R. (1998) Prostate 34, 231-237
39. Ohsumi, T., Ichimura, T., Sugano, H., Omata, S., Isobe, T., and Kuwano, R. (1993) Biochem. J. 294, 465-472
40. Lees, J. A., Buchkovich, K. J., Marshak, D. R., Anderson, C. W., and Harlow, E. (1991) EMBO J. 10, 4279-4290
41. DeCaprio, J. A., Ludlow, J. W., Lynch, D., Furukawa, Y., Griffin, J., Piwnica-Worms, H., Huang, C. M., and Livingston, D. M. (1989) Cell 58, 1085-1095
42. Knudsen, E. S., and Wang, J. Y. J (1996) J. Biol. Chem. 271, 8313-8320
43. Liu, J. J., Chao, J. R., Jiang, M. C., Ng, S. Y., Yen, J. J. and Yang-Yen, H. F. (1995) Mol. Cell. Bio. 15, 3654-3663
44. Kadkol, S. S., Brody, J. R., Pevsner, J., Bai, J., and Pasternack, G. R. (1999) Nature Med. 5, 275-279

What is claimed is:

1. A method of controlling proliferation of a cell population comprising administering at least one agent that inhibits the association of pp32 with hyperphosphorylated Retinoblastoma protein (pRb).

2. The method of claim 1, wherein said agent is administered in an amount sufficient to promote apoptosis within said cell population or to reduce E2F1-mediated transcription.

3. The method of claim 1, wherein said agent that inhibits the association of pp32 with pRb is a peptide.

4. The method of claim 3, wherein said peptide comprises the peptide of SEQ ID NO:3.

5. The method of claim 3, wherein said peptide comprises a fragment of the peptide of SEQ ID NO:3, and wherein said fragment of the peptide of SEQ ID NO:3 inhibits the association of pp32 with phosphorylated Retinoblastoma protein, and wherein amino acid Thr⁸²⁶ of said Retinoblastoma protein is phosphorylated.

6. The method of claim 3, wherein said peptide is an antibody fragment.

7. The method of any one of claims 3 to 6, wherein said peptide further comprises a nuclear localization signal.

8. The method of claim 1, wherein amino acid Thr⁸²⁶ of said Retinoblastoma protein is phosphorylated.

9. The method of claim 1, wherein said cell population comprises tumor cells.

10. The method of claim 10, wherein apoptosis is induced in said tumor cells.

11. The method of claim 1, wherein said agent comprises an expression vector comprising the nucleic acid sequence of SEQ ID NO:4 operably linked to a promoter active in said cell population.

12. The method of claim 11, wherein said expression vector is a gene therapy vector.

13. The method of claim 1, wherein said cell population comprises neoplastic cells.

14. The method of claim 13, wherein said neoplastic cells are prostate cancer cells.

15. The method of claim 13, wherein said neoplastic cells are selected from the group consisting of breast, colon, lung, stomach, and pancreatic cancer cells, leukemias, lymphomas, melanomas and other skin cancer cells, and brain cancer cells including glioblastoma cells.

16. The method of claim 1, wherein said agent comprises a peptidomimetic.

17. A method of screening for agents that inhibit the association of pp32 with pRb, comprising (a) mixing a candidate agent with pp32 and pRb, and (b) measuring the binding of pp32 to pRb.

18. The method of claim 17, wherein at least one of said pp32 or pRb of step (a) is provided in a cell lysate.
